(19) United States
(12) Patent Application Publication
(10) Pub. No.: US 2013/0023056 A1
(43) Pub. Date: Jan. 24, 2013

(54) EARLY DETECTION OF RECURRENT BREAST CANCER USING METABOLITE PROFILING

(71) Applicant: PURDUE RESEARCH FOUNDATION, West Lafayette, IN (US)

(72) Inventors: M. Daniel Raftery, Seattle, WA (US); Vincent Moseti Asiago, Johnston, IA (US); G.A. Nagana Gowda, West Lafayette, IN (US); Leiddy Alvarado, Lafayette, IN (US)

(73) Assignee: PURDUE RESEARCH FOUNDATION, West Lafayette, IN (US)

(21) Appl. No.: 13/624,042

(22) Filed: Sep. 21, 2012

Related U.S. Application Data
(63) Continuation of application No. PCT/US2011/029681, filed on Mar. 23, 2011.
(60) Provisional application No. 61/316,679, filed on Mar. 23, 2010.

(51) Int. Cl. G01N 27/62; C07C 215/40; C07C 53/02; C07D 233/56; G06F 17/18; C07C 229/08; C07C 59/235; C07D 207/16; C07C 229/22; C07C 229/36; C07C 59/01; C07C 229/24

(52) U.S. Cl. 436/90; 562/579; 564/293; 562/609; 548/339.1; 562/573; 562/575; 554/121; 548/535; 562/570; 562/478; 436/129; 702/19

(57) ABSTRACT

A monitoring test for recurrent breast cancer with a high degree of sensitivity and specificity is provided that detects the presence of a panel of multiplicity of biomarkers that were identified using metabolite profiling methods. The test is capable of detecting breast cancer recurrence about a years earlier than current available monitoring diagnostic tests. The panel of biomarkers is identified using a combination of nuclear magnetic resonance (NMR) and two dimensional gas chromatography-mass spectrometry (GC×GC-MS) to produce the metabolite profiles of serum samples. The NMR and GC×GC-MS data are analyzed by multivariate statistical methods to compare identified metabolite signals between samples from patients with recurrence of breast cancer and those from patients having no evidence of disease.

### CROSS-REFERENCE TO RELATED APPLICATION

This application is a continuation of co-pending intentional patent application PCT/US2011/029681, filed on Mar. 23, 2011, and claims benefit of U.S. provisional patent application Ser. No. 61/316,679, filed on Mar. 23, 2010. The entire disclosures of both applications are incorporated herein by reference.

### STATEMENT REGARDING FEDERALLY SPONSORED RESEARCH

This invention was made with United States government support under R01 GM085291 from the National Institute of General Medical Sciences. The United States government has certain rights to this invention.

### TECHNICAL FIELD

The present disclosure generally relates to small molecule biomarkers comprising a panel of metabolite species that is effective for the early detection of breast cancer recurrence, including methods for identifying such panels of biomarkers within biological samples by using a process that combines gas chromatography-mass spectrometry and nuclear magnetic resonance spectrometry.

### BACKGROUND

Breast cancer remains the leading cause of death among women worldwide. It is the second leading cause of death among women in the United States, with nearly 190,000 new cases and 40,000 deaths expected in the year 2010. Although breast cancer survival has improved over the past few decades owing to improved diagnostic screening methods breast cancer often recurs anywhere from 2 to 15 years following initial treatment, and can occur either locally in the same or contralateral breast or as a distant recurrence (metastasis). Recent studies of nearly 3,000 breast cancer patients showed that the recurrence rate 5 and 10 years after completion of adjuvant treatment were 11 percent (“%”) and 20%, respectively. Numerous factors such as stage, grade and hormone receptor status are shown to have association with recurrence. Higher stage tumors often have higher propensity to recur. For example, a recent study reports that 7%, 11% and 13% of recurrence after 5 years for stage I, II and III tumor cases, respectively. In addition, conditions such as lymph node invasion and absence of estrogen receptors are factors in a higher relapse rate and a shorter disease free survival. Studies have shown that early detection of locally recurrent breast cancers can improve survival rate significantly.

Common methods for routine surveillance of recurrent breast cancer include periodic mammographic examinations, self-examination or physician-performed physical examination and blood tests. The performances of such tests are poor, and extensive investigations for surveillance have not proven effective. Often, mammography misses small local recurrences or leads to false positives, resulting in low sensitivity and specificity, and unnecessary biopsies. In view of the unmet need for more sensitive and earlier detection methods, the last decade or so has witnessed the development of a number of new approaches for detecting recurrent breast cancer and monitoring disease progression using blood based tumor markers or genetic profiles. The in vitro diagnostic (“IVD”) markers include carcinoembryonic antigen (“CEA”), cancer antigen (“CA”) 15-3, CA 27.29, tissue polypeptide antigen (“TPA”), and tissue polypeptide specific antigen (“TPS”). Such molecular markers are thought to be promising since the outcome of the diagnosis based on these markers is independent of the expertise and experience of the clinicians and it potentially avoids sampling errors commonly associated with conventional pathological tests, such as histopathology. However, currently these markers tack the desired sensitivity and specificity, and often respond late to recurrence, underscoring the need for alternative approaches.

Up to nearly 50% improvement in the relative survival of patients can be achieved by detecting the recurrence at a clinically asymptomatic phase, showing the need for a reliable test that is based on biomarkers that are indicative of secondary tumor cell proliferation. However, the performance of the commercially available non-invasive tests based on circulating tumor markers such as carcinoembryonic antigen and cancer antigens is too poor to be of significant value for improving early detection. This is because the levels of these markers are also elevated in numerous other malignant and non-malignant conditions unconnected with breast cancer. Considering such limitations, the American Society of Clinical Oncologists (ASCO) guidelines recommend the use of these markers only for monitoring patients with metastatic disease during active therapy in conjunction with numerous other examinations and investigations.

Metabolite profiling (or metabolomics), can detect disease based on a panel of small molecules derived from the global or targeted analysis of metabolic profiles of samples such as blood and urine. Metabolite profiling uses high-resolution analytical methods such as nuclear magnetic resonance (NMR) spectroscopy and mass spectrometry (MS) for the quantitative analysis of hundreds of small molecules (less than ˜1,000 Da) present in biological samples. Owing to the complexity of the metabolic profile, multivariate statistical methods are extensively used for data analysis. The high sensitivity of metabolite profiles to even subtle stimuli can provide the means to detect the early onset of various biological perturbations in real time.

### SUMMARY OF THE INVENTION

A monitoring test for recurrent breast cancer with a high degree of sensitivity and specificity is provided that detects the presence of a panel of multiplicity of biomarkers that were identified using metabolite profiling methods. The test is capable of detecting breast cancer recurrence about a years earlier than current available monitoring diagnostic tests. The panel of biomarkers is identified using a combination of nuclear magnetic resonance (NMR) and two dimensional gas chromatography-mass spectrometry (GC×GC-MS) to produce the metabolite profiles of serum samples. The NMR and GC×GC-MS data are analyzed by multivariate statistical methods to compare identified metabolite signals between samples from patients with recurrence of breast cancer and those from patients having no evidence of disease.

In a preferred embodiment, a method is disclosed for detecting a panel of a multiplicity of predetermined metabolic biomarkers that are indicative of the recurrence of breast cancer in a subject, comprising obtaining a sample of a biofluid from the subject; analyzing the sample to determine the presence and the amount of each of the metabolic biomarkers in the panel; wherein the presence and the amount of each of the metabolic biomarkers in the panel as a whole are indicative of the recurrence of breast cancer in a subject. Typically the biofluid is blood, plasma, serum, sweat, saliva, sputum, or urine. Preferably the biofluid is serum.

In a preferred embodiment, the panel of a multiplicity of metabolic biomarkers consists of at least seven compounds selected from the group consisting of 3-hydroxybutyrate acetoacetate, alanine, arginine, asparagine, choline, creatinine, glucose, glutamic acid, glutamine, glycine, formate, histidine, isobutyrate, isoleucine, lactate, lysine, methionine, N-acetylaspartate, proline, threonine, tyrosine, valine, 2-hydroxy butanoic acid, hexadecanoic acid, aspartic acid, 3-methyl-2-hydroxy-2-pentenoic acid, dodecanoic acid, 1,2,3, trihydroxypropane, beta-alanine, alanine, phenylalanine, 3-hydroxy-2-methyl-butanoic acid 9,12-octadecadienoic acid, acetic acid, N-acetylglycine, glycine, nonanedioic acid, nonanoic acid, and pentadecanoic acid.

In another preferred embodiment, the panel consists of 3-hydroxybutyrate, acetoacetate, alanine, arginine, choline, creatinine, glutamic acid, glutamine, formate, histidine, isobutyrate, lactate, lysine, proline, threonine, tyrosine, valine, hexadecanoic acid, aspartic acid, dodecanoic acid, alanine, phenylalanine, 3-hydroxy-2-methyl-butanoic acid, 9,12 octadecadienoic acid, acetic acid, N-acetylglycine, nonanedioic acid, and pentadecanoic acid.

In a further preferred embodiment, the panel consists of 3 hydroxybutyrate, choline, glutamic acid, formate, histidine, lactate, proline, tyrosine, 3 hydroxy-2-methyl-butanoic acid, N-acetylglycine, and nonanedioic acid. In another preferred embodiment, the panel consists of choline, glutamic acid, formate, histidine, proline, 3 hydroxy-2-methyl-butanoic acid, N-acetylglycine, and nonanedioic acid. In yet another preferred embodiment, the panel consists of 3-hydroxybutyrate, choline, formate, histidine, lactate, proline, and tyrosine.

In a preferred embodiment the metabolic biomarkers in the panel are determined by obtaining samples of biofluid from subjects with known breast cancer status; measuring one or more metabolite species in the samples of by subjecting the sample to nuclear magnetic resonance measurements; measuring one or amore metabolite species in the samples of by subjecting the sample to mass spectrometry measurements; analyzing the results of the nuclear magnetic resonance measurements and the results of the mass spectrometry measurements to produce spectra containing individual spectral peaks representative of the one or more metabolite species contained within the sample; subjecting the spectra to multivariate statistical analysis to identify one or more metabolite species contained within the sample; and determining which metabolic species are correlated, with a given breast cancer status.

In another preferred embodiment, a method is disclosed for detecting secondary tumor cell proliferation in a mammalian subject comprising: obtaining a sample of a biofluid from the subject; analyzing the sample to determine the presence and the amount of each of the metabolic biomarkers in a panel of predetermined biomarkers; wherein the presence and the amount of each of the metabolic biomarkers in the panel as a whole are indicative of secondary tumor cell proliferation in a mammalian subject. Typically the biofluid is blood, plasma, serum, sweat, saliva, sputum, or urine. Preferably the biofluid is serum.

In a preferred embodiment, the panel of a multiplicity of metabolic biomarkers consists of at least seven compounds selected from the group consisting (of 3-hydroxybutyrate, acetoacetate, alanine, arginine, asparagine, choline, creatine, glucose, glutamic acid, glutamine, glycine, formate, histidine, isobutyrate, isoleucine, lactate, lysine, methionine, N-acetylaspartate, proline threonine, tyrosine, valine, 2-hydroxybutanoic acid, hexadecanoic acid, aspartic acid, 3-methyl-2-hydroxy-2-pentatonic acid, dodecanoic acid, 1,2,3, trihydroxypropane, beta-alanine, alanine, phenylalanine, 3-hydroxy-2-methyl butanoic acid, 9,12-octadecadienoic acid, acetic acid, N-acetylglycine, glycine, nonanedioic acid, nonanoic acid, and pentadecanoic acid. In another preferred embodiment, the panel consists of 3-hydroxybutyrate, acetoacetate, alanine, arginine, choline, creatinine, glutamic acid, glutamine, formate, histidine, isobutyrate, lactate, lysine, proline, threonine, tyrosine, valine, hexadecanoic acid, aspartic acid, dodecanoic acid, alanine, phenylalanine, 3-hydroxy-2-methyl-butanoic acid, 9,12 octadecadienoic acid, acetic acid, N-acetylglycine, nonanedioic acid, and pentadecanoic acid.

In a further preferred embodiment, the panel consists of 3 hydroxybutyrate, choline, glutamic acid, formate, histidine, lactate, proline, tyrosine, 3 hydroxy-2-methyl-butanoic acid, N-acetylglycine, and nonanedioic acid, in another preferred embodiment, the panel consists of choline, glutamic acid, formate, histidine, proline, 3 hydroxy-2-methyl-butanoic acid, N-acetylglycine, and nonanedioic acid. In yet another preferred embodiment, the panel consists of 3-hydroxybutyrate, choline, formate, histidine, lactate, proline, and tyrosine.

In a preferred embodiment the metabolic biomarkers in the panel are determined by obtaining samples of biofluid from subjects with known secondary tumor cell proliferation; measuring one or more metabolite species in the samples of by subjecting the sample to nuclear magnetic resonance measurements; measuring one or more metabolite species in the samples of by subjecting the sample to mass spectrometry measurements; analyzing the results of the nuclear magnetic resonance measurements and the results of the mass spectrometry measurements to produce spectra containing individual spectral peaks representative of the one or more metabolite species contained within the sample; subjecting the spectra to multivariate statistical analysis to identify the at least one or more metabolite species contained within the sample; and determining which metabolic species are correlated with secondary tumor cell proliferation.

In another preferred embodiment, a method is disclosed for detecting the recurrence breast cancer status within a biological sample, comprising: measuring one or more metabolite species within the sample by subjecting the sample to a combined nuclear magnetic resonance and mass spectrometry analysis, the analysis producing a spectrum containing individual spectral peaks representative of the one or more metabolite species contained within the sample; subjecting the individual spectral peaks to a statistical pattern recognition, analysis to identify the at least one or more metabolite species contained within the sample, and correlating the measurement of other one or more metabolite species with a breast cancer status. Preferably, the one or multiple metabolite species is selected from the group consisting of 2-methyl,3-hydroxy butanoic acid; 3-hydroxybutyrate; choline; formate; histidine; glutamic acid; N-acetyl-glycine; nonanedenoic acid; proline; threonine; tyrosine; and combinations thereof. Typically the sample comprises a biofluid, preferably serum. Typically the mass spectrometry analysis comprises a two-dimensional gas chromatography coupled mass spectrometry analysis.

In another preferred embodiment, the invention provides a panel of biomarkers for detecting breast cancer, comprising at least one metabolite species or parts thereof, selected from the group consisting of consisting of 2-methyl,3-hydroxy butanoic acid; 3-hydroxybutyrate; choline; formate; histidine; glutamic acid; N-acetyl-glycine; nonanedenoic acid; proline; threonine; tyrosine; and combinations thereof.

### BRIEF DESCRIPTION OF THE DRAWINGS

The above-mentioned aspects of the present teachings and the manner of obtaining them will become more apparent and the teachings will be better understood by reference to the following description of the embodiments taken in conjunction with the accompanying drawings, in which corresponding reference characters indicate corresponding parts throughout the several views.

FIG. 1A is a flow chart describing one embodiment of a method of biomarker selection, model development, and validation. The samples were split into a training set consisting of NED (n=141) and recurrence samples (n=49) near the time of diagnosis and post diagnosis, and a testing set of samples consisting of pre-diagnosis recurrence samples. The training set of samples were divided into 5 cross validation groups of patients. Logistic regression was used for biomarker selection using 5 fold cross validation. Model building used partial least squares discriminant analysis (PLS-DA) modeling with leave one out internal cross validation. Validation was performed on the prediagnosis samples. FIG. 1B is a flow chart describing another embodiment of biomarker selection, model development, and validation. The samples were randomly split into a training set (n=140, 66 recurrence samples and 74 NED) samples) and testing set (n=117 samples, 50 recurrence samples and 50 NED samples). Variable selection was performed using logistic regression, and a predictive model was constructed based on 7 biomarkers identified in NMR studies and 4 biomarkers identified in GC studies.

FIG. 2A shows a typical 500 MHz one dimension ¹H NMR spectrum, FIG. 2 two dimension GC×GC/TOF-MS total ion current (TIC) contour plot spectrum (without solvent) from a post recurrence breast cancer patient.

FIG. 3A-F shows a validation procedure for MS biomarkers: 3A is a three dimension GC×GC-TOF total ion current (TIC) surface plot chromatogram; 3B is a typical one dimension TIC GC×GC-TOF chromatogram; 3C shows the selected metabolite (glutamic acid) based, on the chromatogram for the selected ion peak at m/z 432, 3D shows a mass spectrum of glutamic acid from an NED patient; 3E shows the mass spectrum for glutamic acid from a patient with recurrent breast cancer; and 3F shows a mass spectrum for glutamic acid for commercial sample of that metabolite.

FIG. 4A-K shows box and whisker plots illustrating the discrimination between post plus within recurrence (“Recurrence”) versus NED patient for all samples for the 7 NMR and the 4 GC×GC/MS markers, expressed as relative peak integrals. The horizontal line in the mid portion of the box represents the mean while the bottom and top boundaries of the boxes represents 25^{th} and 75^{th} percentiles respectively. The lower and upper whiskers represent the minimum and maximum values respectively, while the open circles represent outliers. The y-axis provides relative peak integrals as described in the Methods section. FIG. 4A is based on NMR data for formate. FIG. 4B is based on NMR data for histidine. FIG. 4C is based on NMR data for proline. FIG. 4D is based on NMR data for choline. FIG. 4E is based on NMR data for tyrosine. FIG. 4F is based on NMR data for 3-hydroxybutyrate. FIG. 4G is based on NMR data for lactate. FIG. 4H is based on GC×GC/MS data for glutamate. FIG. 4I is based, on GC×GC/MS data for N-acetylglycine FIG. 4J is based on GC×GC/MS data for 3-hydroxy-2-methyl-butanoic acid. FIG. 4K is based on GC×GC/MS data for nonanedioic acid.

FIG. 5A-R shows box and whisker plots illustrating the discrimination between post plus within recurrence (“Recurrence”) versus NED patient for all samples for additional markers, expressed as relative peak integrals. The horizontal line in the mid portion of the box represents the mean while the bottom and top boundaries of the boxes represents 25^{th} and 75^{th} percentiles respectively. The lower and upper whiskers represent the minimum and maximum values respectively, while the open circles represent outliers. The y-axis provides relative peak integrals as described in the Methods section. FIG. 5A is based on NMR data for arginine. FIG. 5B is based on GC×GC/MS data for dodecanoic acid. FIG. 5C is based on NMR data for alanine. FIG. 5D is based on GC×GC/MS data for alanine. FIG. 5E is based on NMR data for phenylalanine. FIG. 5F is based on GC×GC/MS data for phenylalanine. FIG. 5G is based on GC×GC/MS data for aspartic acid, FIG. 5H is based on NMR data for glutamate. FIG. 5I is based on NMR data for threonine. FIG. 5J is based on NMR data for valine. FIG. 5K is based on NMR data for acetoacetate. FIG. 5L is based on NMR data for lysine. FIG. 5M is based on NMR data for Creatinine. FIG. 5N is based on NMR data for isobutyrate. FIG. 5O is based on GC×GC/MS data for hexadecanoic acid. FIG. 5P is based on GC×GC/MS data for 9,12-octadecadienoic acid. FIG. 5Q is based on GC×GC/MS data for pentadecanoic acid. FIG. 5R is based on GC×GC/MS data for acetic acid.

FIG. 6A shows a ROC curve generated from the PLS-DA model illustrated in FIG. 1A and described below, using data from Post and Within (=“Recurrence”) samples versus data from NED samples, and the performance of CA 27.29 on the same samples. FIG. 6B shows box-and-whisker plots for the two sample classes, showing discrimination of Recurrence samples from the samples for the NED patients by using the model-predicted scores. FIG. 6C shows a ROC curve generated from the PLS-DA prediction model by using the testing sample set based on the second statistical approach illustrated in FIG. 1B. FIG. 6D shows box-and-whisker plots for the two sample classes, showing discrimination of Recurrence samples from the samples from the NED patients by using the predicted scores from the testing set.

FIG. 7A shows the percentage of recurrence patients correctly identified using the 11 biomarker model (BCR Profile 1, filled squares) as a function of time for all recurrence patients using a cutoff threshold of 48, compared to the percentage of recurrence patients correctly identified using the CA 27.29 test (filled triangles). FIG. 7B shows the percentage of NED patients correctly identified using the 11 biomarker model (filled squares) as a function of time using a cutoff threshold of 48, compared to the percentage of NED patients correctly identified using the CA 27.29 test (filled triangles), FIG. 7C shows the percentage of recurrence patients correctly identified using the 11 biomarker model (filled squares) as a function of time for all recurrence patients using a cutoff threshold of 54, compared to the percentage of recurrence patients correctly identified using the CA 27.29 test (filled triangles). FIG. 7D shows the percentage of NED patients correctly identified using the 11 biomarker model (filled squares) as a function of time using a cutoff threshold of 54, compared to the percentage of NED patients correctly identified using the CA 27.29 test (filled triangles).

FIGS. 8A and 8B show the percentage of recurrence patients correctly identified as recurrence based on their estrogen receptor (ER) status (FIG. 8A) and progesterone receptor (PR) status (FIG. 8B) as a function of time using the same 11 biomarker model (BCR. Profile 1) and a cutoff threshold of 48. In FIG. 8A, ER minus status is indicated by the filled triangles and ER plus status is indicated by the filled squares. In FIG. 8B, PR minus status is indicated by the filled triangles and PR plus status is indicated by the filled squares.

FIGS. 9A-9D show ROC curves generated from the prediction model using the training set (FIG. 9A) and the testing set (FIG. 9B) using the statistical approach illustrated in FIG. 1B. Box and whisker plots thr the two sample classes showing discrimination between Recurrence samples from NED samples using the predicted scores from the training set (FIG. 9C) and testing set (FIG. 9D).

FIG. 10 is a summary of the altered metabolism pathways for metabolites that showed significant statistical differences between breast cancer patients with recurrence of the cancer and those with no evidence of disease (NED). The metabolites shown outlined with a solid line were down-regulated in recurrence patients while those shown outlined with a dashed line were up-regulated. In addition to the 11 metabolites used in the metabolite profile, a number of the other, related metabolites from Table 2 and FIGS. 4 and 5 are also shown in FIG. 10.

### DETAILED DESCRIPTION OF EXEMPLARY EMBODIMENTS

In one preferred embodiment, a monitoring test for recurrent breast cancer that was developed using metabolite profiling methods is disclosed. Using a combination of nuclear magnetic resonance (NMR) and two-dimensional gas chromatography-mass spectrometry (GC×GC-MS) methods, we analyzed the metabolite profiles of 257 retrospective serial serum samples from 56 previously diagnosed and surgically treated breast cancer patients. One hundred sixteen of the serial samples were from 20 patients with recurrent breast cancer, and 141 samples were from 36 patients with no clinical evidence of the disease during ˜6 years of sample collection. NMR and GC×GC-MS data were analyzed by multivariate statistical methods to compare identified metabolite signals between the recurrence samples and those with no evidence of disease, producing a set of 40 biomarkers (Table 2, below). A subset of eleven metabolite markers (seven from NMR and four from GC×GC-MS) was selected from an analysis of all patient samples by using logistic regression and 5-fold cross-validation. A partial least squares discriminant analysis model, built using these markers with leave-one-out cross-validation provided a sensitivity of 86% and a specificity of 84% (area under the receiver operating characteristic curve=0.88). Strikingly, 55% of the patients could be correctly predicted to have recurrence more than a year (13 months ort average) before the recurrence was clinically diagnosed, representing a large improvement over the current breast cancer-motoring assay CA 27.29.

The embodiments of the present disclosure described below are not intended to be exhaustive or to limit the disclosure to the precise forms disclosed in the following detailed description. Rather, the embodiments are chosen and described so that others skilled in the art may appreciate and understand the principles and practices of the present disclosure.

Unless defined otherwise, all technical and scientific terms used herein have the meaning commonly understood by a person skilled in the art to which this invention belongs.

As used herein, “metabolite” refers to any substance produced or used during all the physical and chemical processes within the body that create and use energy, such as: digesting food and nutrients, eliminating waste through urine and feces, breathing, circulating blood, and regulating temperature. The term “metabolic precursors” refers to compounds from which the metabolites are made. The term “metabolic products” refers to any substance that is part of a metabolic pathway (e.g. metabolite, metabolic precursor).

As used herein, “biological sample” refers to a sample obtained from a subject. In preferred embodiments, biological sample can be selected, without limitation, from the group of biological fluids (“biofluids”) consisting of blood, plasma, serum, sweat, saliva, including sputum, urine, and the like. As used herein, “serum” refers to the fluid portion of the blood obtained after removal of the fibrin clot and blood cells, distinguished from the plasma in circulating blood. As used herein, “plasma” refers to the fluid, non-cellular portion of the blood, as distinguished from the serum, which is obtained after coagulation.

As used herein, “subject” refers to any warm-blooded animal, particularly including a member of the class Mammalia such as, without limitation, humans and non-human primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex and, thus, includes adult and newborn subjects, whether male or female.

As used herein, “detecting” refers to methods which include identifying the presence or absence of substance(s) in the sample, quantifying the amount of substance(s) in the sample, and/or qualifying the type of substance. “Detecting” likewise refers to methods which include identifying the presence or absence of breast cancer tissue or breast cancer recurrence in a subject.

“Mass spectrometer” refers to a gas phase ion spectrometer that measures a parameter that can be translated into mass-to-charge ratios of gas phase ions. Mass spectrometers generally include an ion source and a mass analyzer. Examples of mass spectrometers are time-of-flight, magnetic sector, quadrupole filter, ion trap, on cyclotron resonance, electrostatic sector analyzer and hybrids of these. “Mass spectrometry” refers to the use of a mass spectrometer to detect gas phase ions.

The terms “comprises,” “comprising,” and the like are intended to have the broad meaning ascribed to them in U.S. Patent Law and can mean “includes,” “including” and the like.

It is to be understood that this invention is not limited to the particular component parts of a device described or process steps of the methods described, as such devices and methods may vary. It is also to be understood that the terminology used herein is for purposes of describing particular embodiments only, and is not intended to be limiting. As used in the specification and the appended claims, the singular forms “a,” “an,” and “the” include plural referents unless the context clearly indicates otherwise.

The present disclosure provides a monitoring test based on a panel of selected biomarkers that have been selected as being effective, in detecting the early recurrence of breast cancer. The test has a high degree of clinical sensitivity and clinical specificity and is capable of detecting breast cancer recurrence at a much earlier time point than current monitoring diagnostics. The test is based on biological sample classification methods that utilize a combination of nuclear magnetic resonance (“NMR”) and mass spectrometry (“MS”) techniques. More particularly, the present teachings take advantage of the combination of NMR and two-dimensional gas chromatography-mass spectrometry (“GC×GC-MS”) to identify small molecule biomarkers comprising a set of metabolite species found in patient serum samples. Panels of these identified biomarkers have been found to be effective in detecting recurrent breast cancer at an early stage by comparing identified metabolite signals between recurrence samples and no evidence of disease samples, providing an indication of recurrence more than a year earlier than presently available diagnostic tests or clinical diagnosis.

Metabolite profiling utilizes high-throughput analytical methods such as nuclear magnetic resonance spectroscopy and mass spectroscopy for the quantitative analysis of hundreds of small molecules (less than ˜1000 Daltons) present in biological samples. Owing to the complexity of the metabolic profile, multivariate statistical methods are extensively used for data analysis. The high sensitivity of metabolite profiles to even subtle stimuli can provide the means to detect the early onset of various biological perturbations in real time.

In the present study, the metabolite profiling method was used to determine and select metabolites that are sensitive to recurrent breast cancer and are detected in serum samples. A combination of NMR and two dimensional gas chromatography resolved MS (“2D GC-MS”) methods were utilized to build and validate a model for early breast cancer recurrence detection based on a set of 257 retrospective serial serum samples. The performance of the derived 11 metabolite biomarkers selected for the model compared very favorably with the performance of the currently used molecular marker, CA 27.29, indicating that metabolite profiling methods promise a sensitive test for follow-up surveillance of treated breast cancer patients. In particular, over 60% of the recurring patients could be identified more than 10 months prior to their detection by clinical diagnosis. The resulting test provides a sensitive and specific model for the early detection of recurrent breast cancer

While this metabolite profile was discovered using a platform of NMR and MS methods, one of ordinary skill in the art will recognize that these identified biomarkers can be detected by alternative methods of suitable sensitivity, such as HPLC, immunoassays, enzymatic assays or clinical chemistry methods.

In one embodiment of the invention, samples may be collected from individuals over a longitudinal period of time. Obtaining numerous samples from an individual over a period of time can be used to verify results from earlier detections and/or to identify an alteration in marker pattern as a result of, for example, pathology.

In one embodiment of the invention, the samples are analyzed without additional preparation and/or separation procedures. In another embodiment of the invention, sample preparation and/or ration can involve, without limitation, any of the following procedures, depending on the type of sample collected and/or types of metabolic products searched: removal of high abundance polypeptides (e.g., albumin, and transferrin); addition of preservatives and calibrants, desalting of samples; concentration of sample substances; protein digestions; and fraction collection. In yet another embodiment of the invention, sample preparation techniques concentrate information-rich metabolic products and deplete polypeptides or other substances that would carry little or no information such as those that are highly abundant or native to serum.

In another embodiment of the invention, sample preparation takes place in a manifold or preparation/separation device. Such a preparation/separation device may, for example, be a microfluidics device, such as a cassette. In yet another embodiment of the invention, the preparation/separation device interfaces directly or indirectly with a detection device. Such a preparation/separation device may, for example, be a fluidics device.

In another embodiment of the invention, the removal of undesired polypeptides (e.g., high abundance, uninformative, or undetectable polypeptides) can be achieved using high affinity reagents, high molecular weight filters, column purification ultracentrifugation and/or electrodialysis. High affinity reagents include antibodies that selectively bind to high abundance polypeptides or reagents that have a specific pH, ionic value, or detergent strength. High molecular weight filters include membranes that separate molecules on the basis of size and molecular weight. Such filters may further employ reverse osmosis, nanofiltration, ultrafiltration and microfiltration.

Ultracentrifugation constitutes another method for removing undesired polypeptides. Ultracentrifugation is the centrifugation of a sample at about 60,000 rpm while monitoring with an optical system the sedimentation (or lack thereof) of particles. Finally, electrodialysis is an electromembrane process in which ions are transported through ion permeable membranes from one solution to another under the influence of a potential gradient. Since the membranes used in electrodialysis have the ability to selectively transport ions having positive or negative charge and reject ions of the opposite charge, electrodialysis is useful for concentration, removal, or separation of electrolytes.

In another embodiment of the invention, the manifold or microfluidics device perms electrodialysis to remove high molecular weight polypeptides or undesired polypeptides. Electrodialysis can be used first to allow only molecules under approximately 35 30 kD to pass through into a second chamber. A second membrane with a very small molecular weight cutoff (roughly 500 D) allows smaller molecules to exit the second chamber.

Upon preparation of the samples, metabolic products of interest may be separated in another embodiment of the invention. Separation can take place in the same location as the preparation or in another location. In one embodiment of the invention, separation occurs in the same microfluidics device where preparation occurs, but in a different location on the device. Samples can be removed from an initial manifold location to a microfluidics device using various means, including an electric field. In another embodiment of the invention, the samples are concentrated during their migration to the microfluidics device using reverse phase beads and an organic solvent elution such as 50% methanol. This elutes the molecules into a channel or a well on a separation device of a microfluidics device.

Chromatography constitutes another method for separating subsets of substances. Chromatography is based on the differential absorption and elution of different substances. Liquid chromatography (LC), for example, involves the use of fluid carrier over a non-mobile phase. Conventional LC columns have an in inner diameter of roughly 4.6 mm and a flow rate of roughly 1 ml/min. Micro-LC has an inner diameter of roughly 1.0 mm and a flow rate of roughly 40 μl/min. Capillary LC utilizes a capillary with an inner diameter of roughly 300 im and a flow rate of approximately 5 μl/min. Nano-LC is available with an inner diameter of 50 μm-1 mm and flow rates of 200 nl/min. The sensitivity of nano-LC as compared to HPLC is approximately 3700 fold. Other types of chromatography suitable for additional embodiments of the invention include, without limitation, thin-layer chromatography (TLC), reverse-phase chromatography, high-performance liquid chromatography (HPLC), and gas chromatography (GC).

In another embodiment of the invention, the samples are separated using capillary electrophoresis separation. This will separate the molecules based on their electrophoretic mobility at a given phi (or hydrophobicity), in another embodiment of the invention, sample preparation and separation are combined using microfluidics technology. A microfluidic device is a device that can transport liquids including various reagents such as analytes and elutions between different locations using microchannel structures.

Suitable detection methods are those that have a sensitivity for the detection of an analyte in a biofluid sample of at least 50 μM. In certain embodiments, the sensitivity of the detection method is at least 1 μM. In other embodiments, the sensitivity of the detection method is at least 1 nM.

In one embodiment of the invention, the sample may be delivered directly to the detection device without preparation and/or separation beforehand. In another embodiment of the invention, once prepared and/or separated, the metabolic products are delivered to a detection device, which detects them in a sample. In another embodiment of the invention, metabolic products in elutions or solutions are delivered to a detection device by electrospray ionization (ESI). In yet another embodiment of the invention, nanospray ionization (NSI) is used. Nanospray ionization is a miniaturized version of ESI and provides low detection limits using extremely limited volumes of sample fluid.

In another embodiment of the invention, separated metabolic products are directed down a channel that leads to an electrospray ionization emitter, which is built into a microfluidic device (an integrated ESI microfluidic device). Such integrated ESI microfluidic device may provide the detection device with samples at flow rates and complexity levels that are optimal for detection. Furthermore, a microfluidic device may be aligned with a detection device for optimal sample capture.

Suitable detection devices can be any device or experimental methodology that is able to detect metabolic product presence and/or level, including, without limitation, IR (infrared spectroscopy), NMR (nuclear magnetic resonance), including variations such as correlation spectroscopy (COSy), nuclear Overhauser effect spectroscopy (NOESY), and rotating frame nuclear Overhauser effect spectroscopy (ROESY), and Fourier Transform, 2-D PAGE technology, Western blot technology, tryptic mapping, in vitro biological assay, immunological analysis, LC-MS (liquid chromatography-mass spectrometry, LC-TOF-MS, LC-MS/MS, and MS (mass spectrometry).

For analysis relying on the application of NMR spectroscopy, the spectroscopy may be practiced as one-, two-, or multidimensional NMR spectroscopy or by other NMR spectroscopic examining techniques, among others also coupled with chromatographic methods (for example, as LC-NMR). In addition to the determination of the metabolic product in question, ¹H-NMR spectroscopy offers the possibility of determining further metabolic products in the same investigative run. Combining the evaluation of a plurality of metabolic products in one investigative run can be employed for so-called “pattern recognition”. Typically, the strength of evaluations and conclusions that are based on a profile of selected metabolites, i.e., a panel of identified biomarkers, is improved compared to the isolated determination of the concentration of a single metabolite.

For immunological analysis, for example, the use of immunological reagents (e.g. antibodies), generally in conjunction with other chemical and/or immunological reagents, induces reactions or provides reaction products which then permit detection and measurement of the whole group, a subgroup or a subspecies of the metabolic product(s) of interest. Suitable immunological detection methods with high selectivity and high sensitivity (10-1000 pg, or 0.02-2 pmoles), e.g., Baldo, B. A., et al. 1991, A Specific, Sensitive and High-Capacity Immunoassay for PAF, Lipids 26(12): 1136-1139), that are capable of detecting 0.5-21 ng/ml of an analyte in a biofluid sample (Cooney, S. J., et al, Quantitation by Radioimmunoassay of PAF in Human Saliva), Lipids 26(12): 1140-1143).

In one embodiment of the invention, mass spectrometry is relied upon to detect metabolic products present in a given sample. In another embodiment of the invention, an ESI-MS detection device. Such an ESI-MS may utilizes a time-of-flight (TOF) mass spectrometry system. Quadrupole mass spectrometry, ion trap mass spectrometry, and Fourier transform ion cyclotron resonance (FTICR-MS) are likewise contemplated in additional embodiments of the invention.

In another embodiment of the invention, the detection device interfaces with a separation/preparation device or microfluidic device, which allows for quick assaying of many, if not all, of the metabolic products in a sample. A mass spectrometer may be utilized that will accept a continuous sample stream for analysis and provide high sensitivity throughout the detection process (e.g., an ESI-MS). In another embodiment of the invention, a mass spectrometer interfaces with one or more electrosprays two or more electrosprays, three or more electrosprays or four or more electrosprays. Such electrosprays can originate from a single or multiple microfluidic devices.

In another embodiment of the invention, the detection system utilized allows for the capture and measurement of most or all of the metabolic products introduced into the detection device. In another embodiment of the invention, the detection system allows for the detection of change in a defined combination (“profile,” “panel,” “ensemble, or “composite”) of metabolic products.

### Working Examples

In the Examples, a combination of NMR and 2D GC×GC-MS methods were used to analyze the metabolite profiles of 257 retrospective serial serum samples from 56 previously diagnosed and surgically treated breast cancer patients, 116 of the serial scrum samples were from 20 patients with recurrent breast cancer and 141 serum samples were from 36 patients with no clinical evidence of the disease during the sample collection period. NMR and GC×GC-MS data were analyzed by multivariate statistical methods to compare identified metabolite signals between the recurrence and no evidence of disease samples. Eleven metabolite markers (7 from NMR and 4 from GC×GC-MS) were selected from an analysis of all patient samples by logistic regression model using 5-fold cross validation. A PLS-DA model built using these markers with leave one out cross validation provided a sensitivity of 86% and a specificity of 84% (AUROC>0.85). Strikingly, over 60% of the patients could be correctly predicted to have recurrence 10 months (on average) before the recurrence was diagnosed clinically, representing a large improvement over the current breast cancer monitoring assay CA 27.29. To the best of our knowledge, this is the first study to develop and pre-validate a prediction model for early detection of recurrent breast cancer based on a metabolic profile. In particular, the combination of two advanced analytical methods, NMR and MS, provides a powerful approach for the early detection of recurrent breast cancer.

### Sample Collection.

Two-hundred fifty-seven serum, samples (each ˜400 microliter (μl) from 56 breast cancer patients were obtained from the M.D. Anderson, Cancer Center (Houston, Tex.). These banked serum samples were collected between 1997 and 2003 with an average of 5 serial time-course samples per patient from female volunteers (ages 40-75) who were breast cancer patients enrolled at M.D. Anderson Cancer Center (Houston, Tex.). Follow-up investigations by oncologists at the M.D. Anderson for breast cancer recurrence were based on a combination of factors including CA 27.29, CEA, and/or CA 125 IVD results, patient symptoms, initial breast cancer stage, hormone receptor and lymph node status. Of the 56 patients, breast cancer recurred in 20, either locally or in a distant organ, and the remaining 36 had no evidence of disease (NED) recurrence during the sampling period as well as 2 years afterward.

A total of 116 serum samples were obtained from recurrent breast cancer patients, which constituted 67 samples collected earlier than 3 months before the recurrence was clinically diagnosed (Pre), 18 samples collected within ±3 months of recurrence (Within), and 31 collected later than 3 months after diagnosed recurrence (Post). The remaining 141 samples represented the cases in which the patient remained NED for at least 2 years beyond their sample collection period. Nearly all samples were evaluated for CA 27.29 values at the time of collection and therefore could be used for comparison. Study samples were maintained at −80° C. from collection until their transfer over dry ice to the evaluation laboratory at Purdue University where they were again stored frozen at −80° C. until this study was conducted. Serum samples and accompanying clinical data were appropriately de-identified before transfer into this study. Table 1 summarizes the clinical parameters and demographic characteristics of the cancer patients.

TABLE 1
Summary of Clinical and Demographic Characteristics of the
Patients Whose Samples Were Used in this Study

| | Control | Recurrence |
|---|---|---|
| Clinical Diagnosis | Samples (Patients) | Samples (Patients) |
| No evidence of disease (NED) | 141 (36) | |
| Pre recurrence (Pre) | — | 67 (20) |
| Within recurrence (Within) | — | 18 (18) |
| Post recurrence (Post) | — | 31 (20) |
| Age, mean (range) | 53 (37-75) | 53 (36-66) |
| Breast cancer stage | | |
| I | 47 (11) | 7 (11) |
| II | 59 (16) | 21 (6) |
| III | 10 (6) | 34 (6) |
| Unknown | 26 (6) | 54 (8) |
| ER status | | |
| ER+ | 65 (15) | 67 (11) |
| ER− | 64 (18) | 33 (7) |
| Unknown | 12 (3) | 16 (2) |
| PR status | | |
| PR+ | 52 (13) | 71 (11) |
| PR− | 77 (20) | 29 (7) |
| Unknown | 12 (3) | 16 (2) |
| CA 27.29 | 140 (36) | 92 (19) |
| Site of recurrence | | |
| Bone | | 37 (6) |
| Breast | | 13 (2) |
| Liver | | 11 (2) |
| Lung | | 10 (6) |
| Skin | | 6 (2) |
| Brain | | 15 (2) |
| Lymph | | 6 (1) |
| Multiple sites | | 18 (3) |

### ¹H NMR Spectroscopy

After thawing, 200 microliter (“μL”) serum was mixed with 330 μL D₂O and 5 μL sodium azide (12.3 nmol). Sample solutions were vortexed for 60 seconds (sec.) and centrifuged for 5 minutes (min.) at 8000 revolutions per minute (RPM). Thereafter, 530 μL aliquots were transferred into standard 5 millimeter (mm) NMR tubes for NMR measurements. An external capillary tube (a glass stem coaxial insert, OD 2 mm) containing 60 μL 0.012% 3-(trimethylsilyl) propionic-(2,2,3,3-d₄) acid sodium salt (“TSP”) solution in D₂O was used as a chemical shift frequency standard (δ=0.00 ppm) and for locking purposes. All NMR experiments were carried out at 25° C. on a Bruker DRX 500 Megahertz (“MHz”) spectrometer equipped with a cryogenic probe and triple-axis magnetic field gradients. Two ¹H NMR spectra were measured for each sample, a standard 1D NOESY (Nuclear Overhauser Effect Spectroscopy) and CPMG (Carr-Purcell-Meiboom-Gill) pulse sequences coupled with water pre-saturation. For each spectrum, 32 transients were collected using 32 k data points and a spectral width of 6000 Hz. An exponential weighting function corresponding to 0.3 Hz line broadening was applied to the free induction decay (FID) before applying Fourier transformation. Each peak was integrated and then normalized using the value of the total NMR spectral intensity (total sum) excluding the water and urea peaks. After phasing and baseline correction using Bruker XWINNMR software version 3.5, the processed data were saved in ASCII format for further analysis.

### GC×GC-MS

Protein precipitation was performed for each sample by mixing 200 μL serum with 400 μL methanol in a 1.5 mL Eppendorf tube. The mixture was briefly vortexed, and then held at −20° C. for 30 min. The samples were centrifuged while still cold at 14,000 RPM for 10 min. The upper layer (supernatant) was transferred into another Eppendorf tube for further use. Chloroform (200 μL) was mixed with the protein pellet and centrifuged at 14,000 RPM for another 10 min. After centrifugation, the aliquot was transferred and combined with the methanol supernatant solution from the previous step. The resultant mixture was lyophilized to remove the solvents for 5 hrs using a Speed Vac (Savant AES2010). Each dried sample was then dissolved in 50 μL of anhydrous pyridine and after a brief vortexing was sonicated for approximately 20 min. Twenty μL of this solution was mixed with 20 μL of the derivatizing reagent MTBSTFA (N-methyl-N-(tert-butyldimethylsilyl, trifluoroacetamide) (Regis, Morton Grove, Ill.). Addition of this derivatizing agent containing an active tert-butyldimethylsilyl group to the mixture activates functional groups such as the hydroxyl, amines or carboxylic acid of the metabolites present in the biological sample. The samples were then incubated at 60° C. for 1 hr to affect the reaction. After derivatization, the solution contents were transferred to a glass GC (auto sampler) vial for the analysis.

Two dimensional GC×GC-MS analysis was performed using a Pegasus 4D system (LECO, St. Joseph, Mich.) consisting of an Agilent 6890 gas chromatograph (Agilent Technologies, Palo Alto, Calif.) coupled to a Pegasus time of flight mass spectrometer. The first dimension chromatographic separation was performed on a DB-5 capillary column (30 m×0.25 mm inner diameter 0.25 μm film thickness). At the end of the first column the eluted samples were frozen by cryotrapping for a period of 4 s and then quickly heated and sent to the second dimension chromatographic column (DB-17, 1 m×0.1 mm inner diameter, 0.10 μm film thickness). The first column temperature ramp began at 50° C. with a hold time of 0.2 min, which was then increased to 300° C. at rate of 10° C./min and held at this temperature for 5 min. The second column temperature ramp was 20° C. higher than the corresponding first column temperature ramp with the same rate and hold time. The second dimension separation time was set for 4 sec. High purity helium was used as a carrier gas at a flow rate of 1.0 mL/min. The temperatures for the inlet and transfer line were set at 280° C., and the ion source was set a 200° C. The detection and filament bias voltages were set to 1600 V and −70 V, respectively.

Mass spectra ranging from 50 to 600 m/z were collected at a rate of 50 Hz. LECO ChromaTOF software (version 4.10) was used for automatic peak detection and mass spectrum deconvolution. The NIST MS database (NIST MS Search 2.0, NIST/EPA/NIH Mass Spectral Library; NIST 2002) was used for data processing and peak matching. Mass spectra of all identified compounds were compared with standard mass spectra in the NIST database (NIST MS Search 2.0, NIST/EPA/NIH Mass Spectral Library; NIST 2002). Further, the identified biomarker candidates were confirmed from the mass spectra and retention times of authentic commercial samples purchased and run under identical experimental conditions.

### Metabolite Identification and Selection

The NMR spectrum from each sample was aligned with reference to the 3-(trimethylsilyl) propionic-(2,2,3,3-d4) (“TSP”) acid sodium salt signal at 0 ppm. Spectral regions within the range of 0.5 to 9.0 ppm were analyzed after excluding the region between 4.5 and 6.0 ppm that contained the residual water peak and urea signal. Twenty-two spectral regions, corresponding to biomarkers, initially identified in a study on early breast cancer detection, were selected as biomarker candidates for further analysis. The statistical significance of each metabolite in the selected regions was determined by calculating the P-values using Student's t-test in the training set. To further enhance the pool of metabolites, 18 additional metabolites were identified for targeted MS analysis based on highest difference in intensity of the peaks between recurrence and NED samples. (Table 2). A software program was developed in-house to extract these metabolite signals from the GC×GC-MS datasets. Based on the input value of m/z and a retention time range, the program integrates chromatography peaks for each metabolite after the metabolite's spectrum was matched to the characteristic experimental mass spectrum from the standard NIST library available in the LECO Chroma TOF software package (v1.61).

The complete set of biomarkers identified using the present method consists of 3-hydroxybutyrate, acetoacetate, alanine, arginine, asparagine, choline, creatinine, glucose, glutamic acid, glutamine, glycine, formate, histidine, isobutyrate, isoleucine, lactate, lysine, methionine, N-acetylaspartate, proline, threonine, tyrosine, valine, 2-hydroxy butanoic acid, hexadecanoic acid, aspartic acid, 3-methyl-2-hydroxy-2-pentenoic acid, dodecanoic acid, 1,2,3, trihydroxypropane, beta-alanine, alanine, phenylalanine, 3 hydroxy-2-methyl-butanoic acid, 9,12-octadecadienoic acid, acetic acid, N-acetylglycine, glycine, nonanedioic acid, nonanoic acid, and pentadecanoic acid (Table 2).

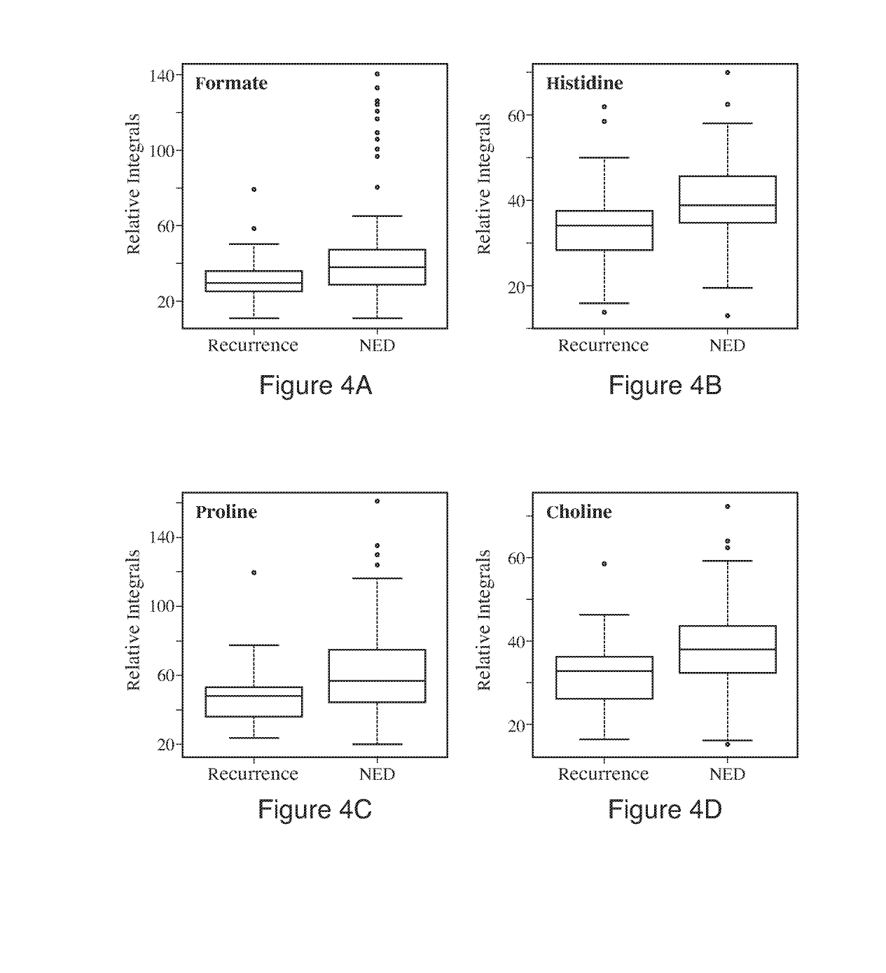

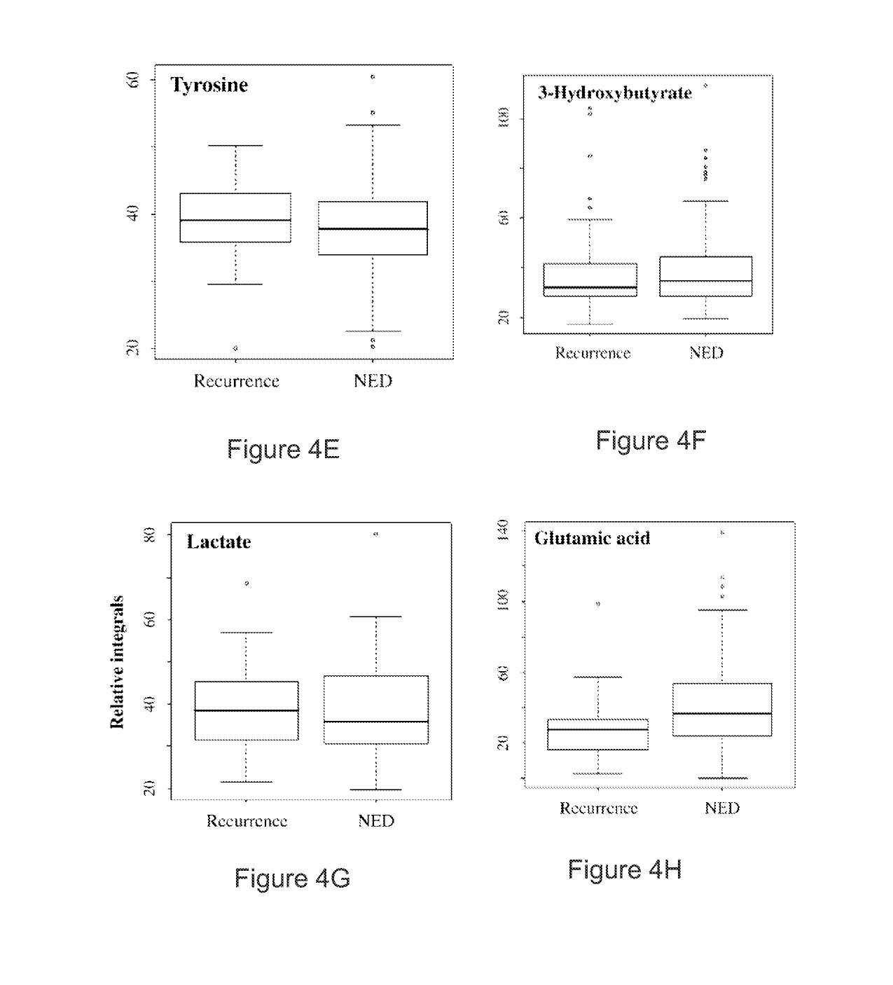

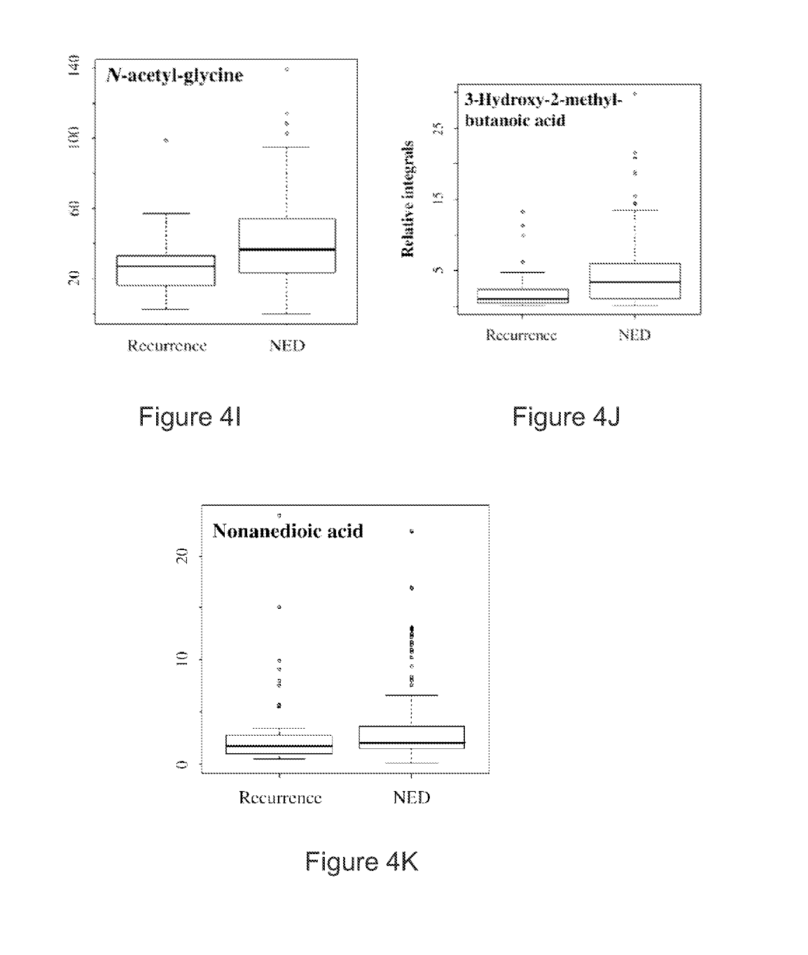

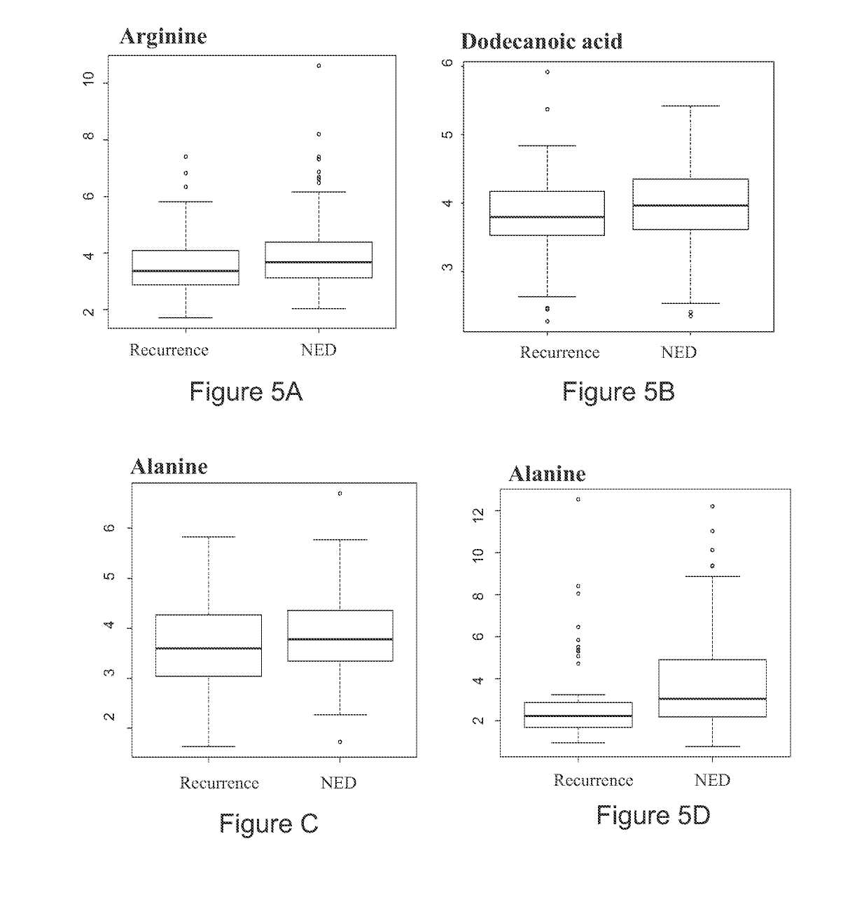

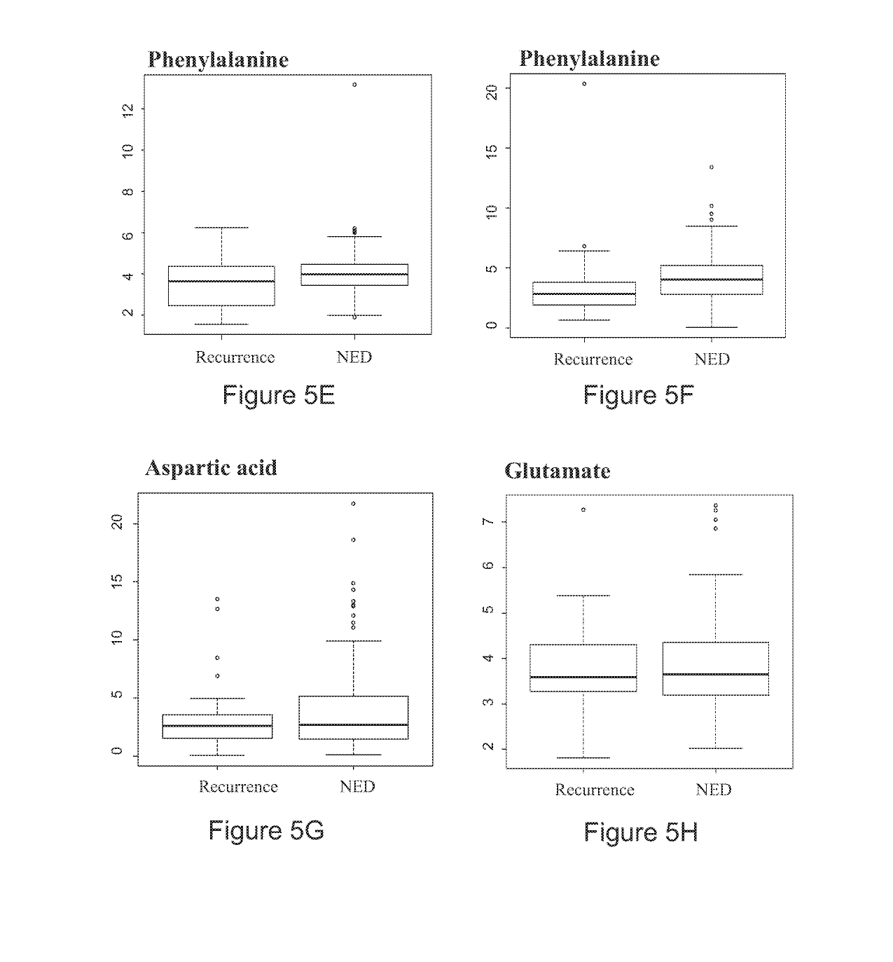

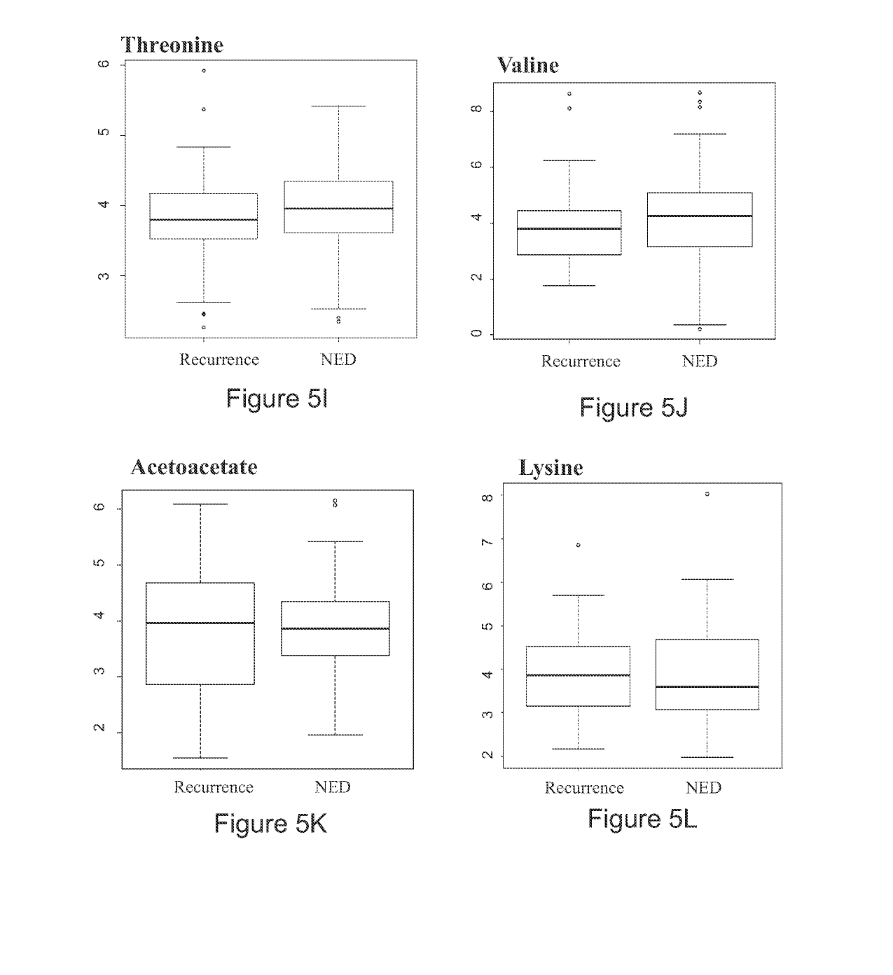

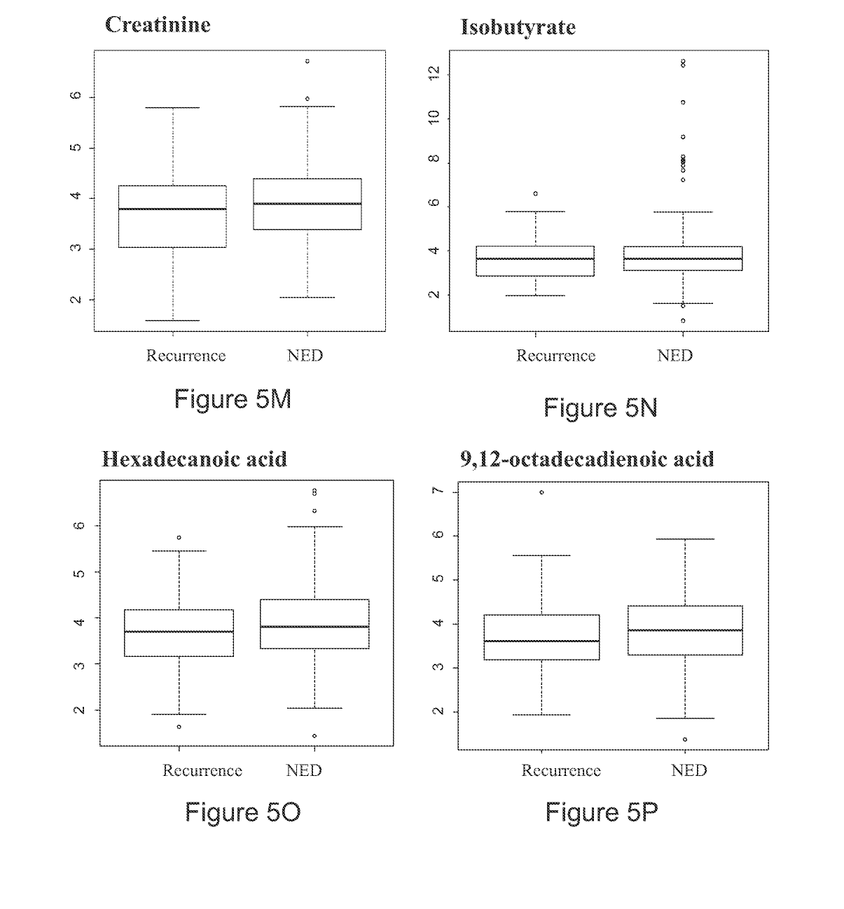

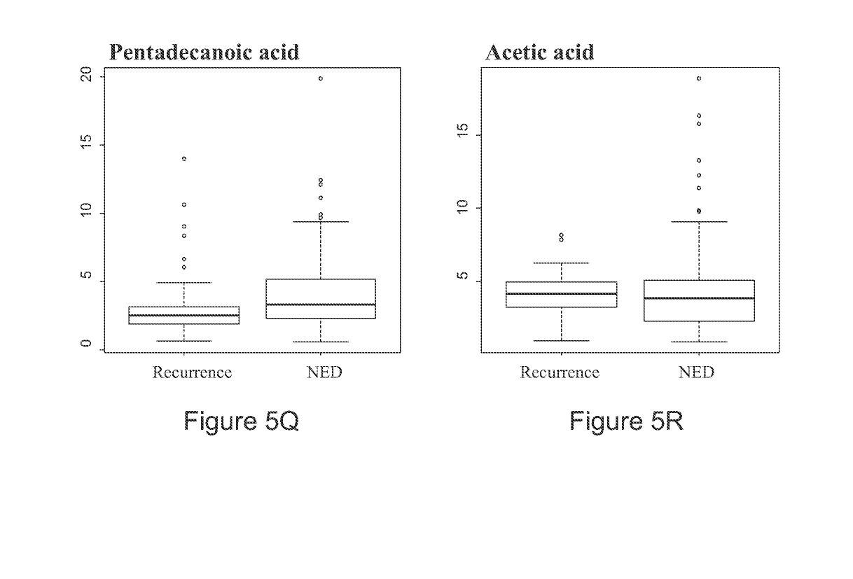

Further analysis was performed on a subset of the biomarkers, as illustrated in the box and whisker plots of FIGS. 4A-4K and FIGS. 5A-5R. This subset of biomarkers consists of 3-hydroxybutyrate, acetoacetate, alanine, arginine, choline, creatinine, glutamic acid, glutamine, formate, histidine, isobutyrate, lactate, lysine, proline, threonine, tyrosine, valine, hexadecanoic acid, aspartic acid, dodecanoic acid, alanine, phenylalanine, 3-hydroxy-2-methyl-butanoic acid, 9,12 octadecadienoic acid, acetic acid, N-acetylglycine, nonanedioic acid, and pentadecanoic acid.

A further subset, or panel, of biomarkers was selected for the development of prediction models and validation of the models, consisting of the metabolites 3-hydroxybutyrate, choline, glutamic acid, formate, histidine, lactate, proline, tyrosine, 3 hydroxy-2-methyl-butanoic acid, N-acetylglycine and nonanedioic acid.

TABLE 2
ALL BIOMARKERS IDENTIFIED FROM NMR ANALYSIS [1-22]
AND GCxGC/MS ANALYSIS [23-40]

| | Metabolite | FIG. | KEGG ID | Pathway |
|---|---|---|---|---|
| 1 | 3-Hydroxybutyrate | 4F | C01089 | Synthesis and degradation of ketone bodies |
| 2 | Acetoacetate | 5K | C00164 | Valine, leucine and isoleucine degradation |
| 3 | Alanine | 5C | C00041 | Alanine, aspartate and glutamate metabolism |
| 4 | Arginine | 5A | C00062 | Arginine and proline metabolism |
| 5 | Asparagine | | C00152 | Alanine, aspartate and glutamate metabolism |
| 6 | Choline | 4D | C00114 | Glycerophospholipid metabolism |
| 7 | Creatinine | 5M | C00791 | Amino acid metabolism |
| 8 | Glucose | | C00031 | Glycolysis and gluconeogenesis |
| 9 | Glutamic acid | 5H | C00025 | D-Glutamine and D-glutamate metabolism |
| 10 | Glutamine | | C00064 | D-Glutamine and D-glutamate metabolism |
| 11 | Glycine | | C00037 | Glycine, serine and threonine metabolism |
| 12 | Formate | 4A | C00058 | Glycoxylate and dicarboxylate metabolism |
| 13 | Histidine | 4B | C00135 | Histidine metabolism |
| 13a | Isobutyrate | 5N | C02632 | Protein digestion and absorption |
| 14 | Isoleucine | | C00407 | Valine, leucine and isoleucine degradation |
| 15 | Lactate | 4G | C00186 | Glycolysis |
| 16 | Lysine | 5L | C00047 | Lysine biosynthesis |
| 17 | Methionine | | C00073 | Cysteine and methionine metabolism |
| 18 | N-Acetylaspartate | | C01042 | Alanine, aspartate and glutamate metabolism |
| 19 | Proline | 4C | C00148 | Arginine and proline metabolism |
| 20 | Threonine | 5I | C00188 | Glycine, serine and threonine metabolism |
| 21 | Tyrosine | 4E | C00082 | Tyrosine metabolism |
| 22 | Valine | 5J | C00183 | Valine, leucine and isoleucine degradation |
| 23 | 2-hydroxy butanoic acid | | C05984 | Propanoate metabolism |
| 24 | Hexadecanoic acid | 5O | C00249 | Fatty acid metabolism |
| 25 | Aspartic acid | 5G | C00049 | Pantothenate and CoA biosynthesis |
| 26 | 3-methyl-2-hydroxy-2-pentenoic | | — | Unknown |
| | acid | | | |
| 27 | Dodecanoic acid | 5B | C02679 | Fatty acid metabolism |
| 28 | L-glutamic acid | 4H | C00025 | D-glutamine and glutamate metabolism |
| 29 | 1,2,3,trihydroxypropane | | C00116 | Galactose metabolism |
| 30 | Beta-alanine | | C00099 | Beta-alanine metabolism |
| 31 | Alanine | 5D | CC00041 | Alanine, aspartate and glutamate metabolism |
| 32 | Phenylalanine | 5E, 5F | C00079 | Phenylalanine metabolism |
| 33 | 3-hydroxy-2 methyl-butanoic acid | 4J | — | Unknown |
| 34 | 9,12-octadecadienoic acid | 5P | C01595 | Linoleic acid metabolism |
| 35 | Acetic acid | 5R | C00033 | Citrate cycle, Pyruvate metabolism |
| 36 | N-acetylglycine | 4I | — | Unknown |
| 37 | Glycine | | C00037 | Glycine serine and threonine metabolism |
| 38 | Nonanedioic acid | 4K | C08261 | Fatty acid metabolism |
| 39 | Nonanoic acid | | C01601 | Unknown |
| 40 | Pentadecanoic acid | 5Q | C16537 | Unknown |

Alternatively, a subset, or panel, of eight biomarkers was selected, consisting of the metabolites choline, glutamic acid, formate, histidine, proline, 3 hydroxy-2-methyl-butanoic acid, N-acetylglycine, and nonanedioic acid.

In other embodiments, a subset, or panel, of seven biomarkers was selected, consisting of the metabolites 3-hydroxybutyrate, choline, formate, histidine, lactate, proline, and tyrosine.

### Development of Prediction Model and Validation

In order to select the metabolites with highest scores for developing the prediction model, samples from NED, post and within recurrence groups were used. Pre-recurrence samples were omitted to avoid any ambiguity in determining the correct disease status prior to clinical diagnosis. Post and within recurrence vs. NED samples were divided into five cross validation (CV) groups. Multivariate analysis using logistic regression model of the 22 NMR and 18 GC×GC/MS detected metabolite signals was applied to 4 CV groups and the resulting model was used to predict the class membership of the 5^{th} CV group. The output of the logistic regression procedure is a ranked set of markers. The best combination of NMR and GC markers that resulted to a model with lowest misclassification error rate and the highest predictive power was retained and used to build final prediction model using all samples.

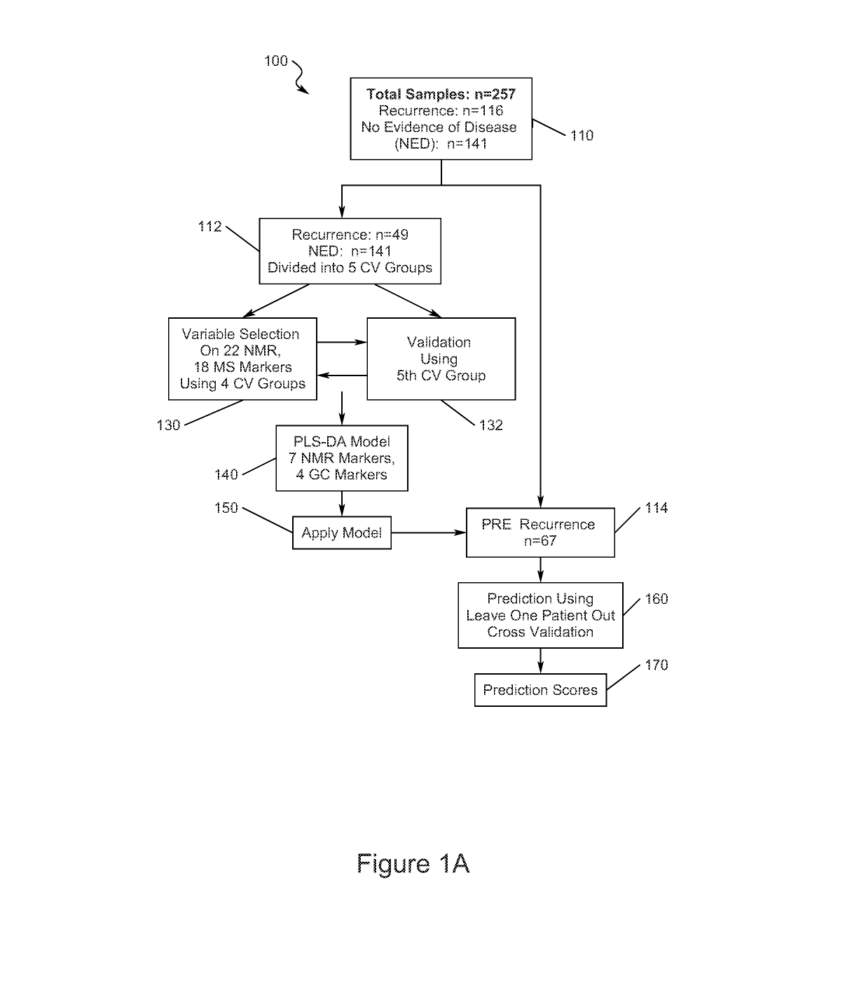

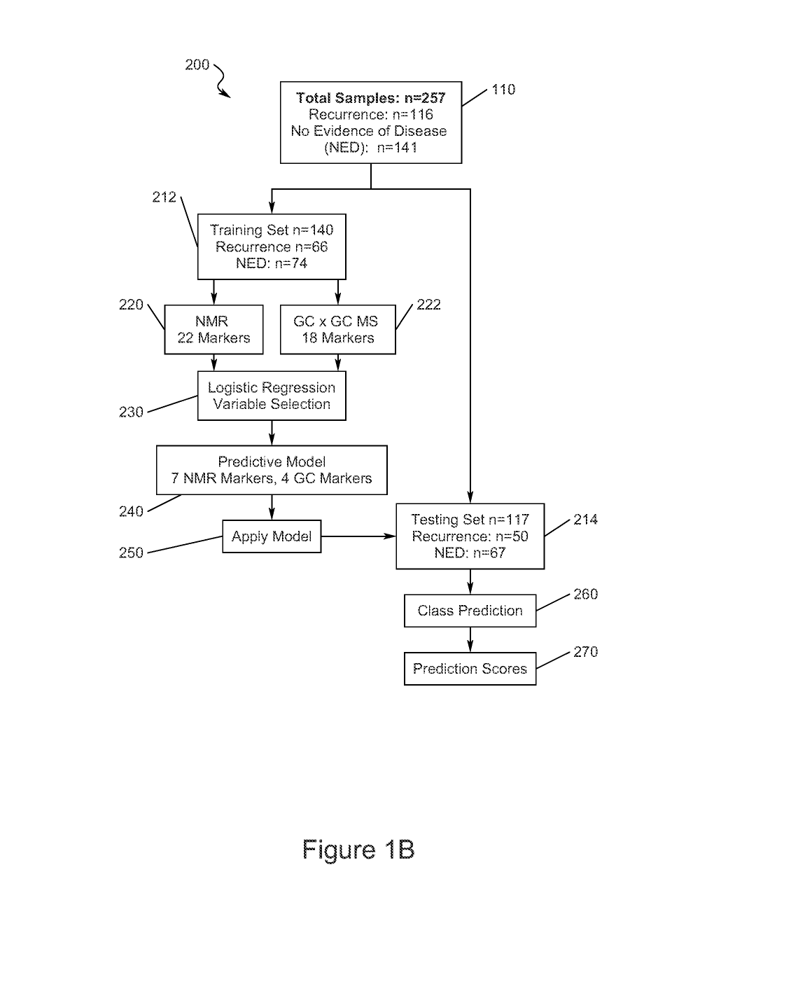

FIG. 1A is a flow chart describing one embodiment of a method 100 of biomarker selection, model development, and validation. A total of 275 serum samples (116 samples from recurrence patients, 141 samples from NED patients were provided, 110. The samples were split into a training set consisting of NED (n=141) and recurrence samples (n=49) near the time of diagnosis and post diagnosis, 112, and a testing set of samples consisting of pre-diagnosis recurrence samples, 114. The training set of samples were divided into 5 cross validation groups of patients, 130 and 132. Logistic regression was used for biomarker selection using 5 fold cross validation. Model building used partial least squares discriminant analysis (PLS-DA) modeling with leave one out internal cross validation 140. Validation was performed by applying the model 150 to the pre-diagnosis samples 114, providing a prediction using leave one patient out cross validation, 160, and yielding prediction sores, 170.

FIG. 1B is a flow chart describing another embodiment of biomarker selection, model development, and validation, 200. A total of 257 serum samples (116 samples from recurrence patients, 141 samples from NED patients were provided, 110. The samples were randomly split into a training set (n=140, 66 recurrence samples and 74 NED samples), 212, and a resting set (n=117 samples, 50 recurrence samples and 50 NED samples), 214. Variable selection was performed using logistic regression, 230, and a predictive model was constructed based on 7 biomarkers identified in NMR studies and 4 biomarkers identified in GC studies, 240. Validation was performed by applying the model 250 to the testing set, 214, providing a class prediction, 260, and yielding prediction scores 270.

Based on their performance, eleven metabolite markers (7 from NMR and 4 GC×GC-MS) were selected for model building. NMR and MS data for these markers were imported into Matlab software (Mathworks, MA) installed with the PLS toolbox (Eigenvector Research, Inc, version 4.0) for PLS-DA modeling. Leave one out cross validation was chosen and the number of latent variables (LV) were selected according to the root mean square error of the cross validation (RMSECV). The R statistical package (version 2.8.0) was used to generate the receiver operating characteristics (ROC) curves. The sensitivity, specificity and the area under the receiver operating characteristic curve (AUROC) of the model was calculated and compared.

The performance of these markers was also assessed based on the time of sample collection, before or after the clinical diagnosis of the recurrence (post recurrence vs. NED within recurrence vs. NED and pre-recurrence vs. NED). The class membership of each sample was determined and compared to the patient's status. The ROC curve was generated and AUROC, sensitivity, and specificity were calculated. The scores from the model were scaled to yield a range of 0-100, and the cutoff vale for recurrence status was determined by a judicious choice between sensitivity and specificity. The performance of the model with reference to the initial stage of the breast cancer, ER/PR status, and the site of recurrence was also assessed.

Finally, the performance of the NMR and MS metabolite markers was also tested by splitting the samples randomly into two parts, training (141 samples) and testing (116 samples) sets and analyzed as illustrated in FIG. 1B. Multivariate logistic regression of the 22 NMR and 18 GC×GC/MS detected metabolites was applied to the training data set to optimize variable selection. Ten-fold cross validation was used during this procedure. The derived model was then validated on the “testing set” of samples, all from different patients than were used for variable selection and model building.

### Analysis of ¹H NMR and GC×GC/MS Spectra

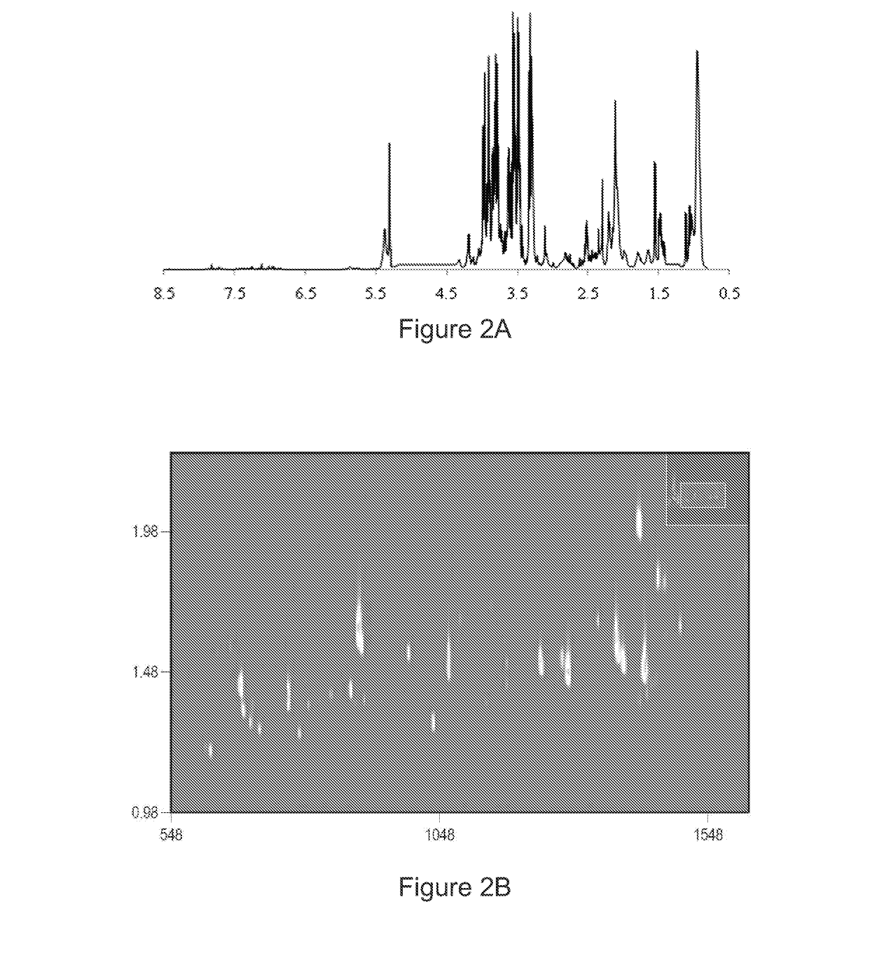

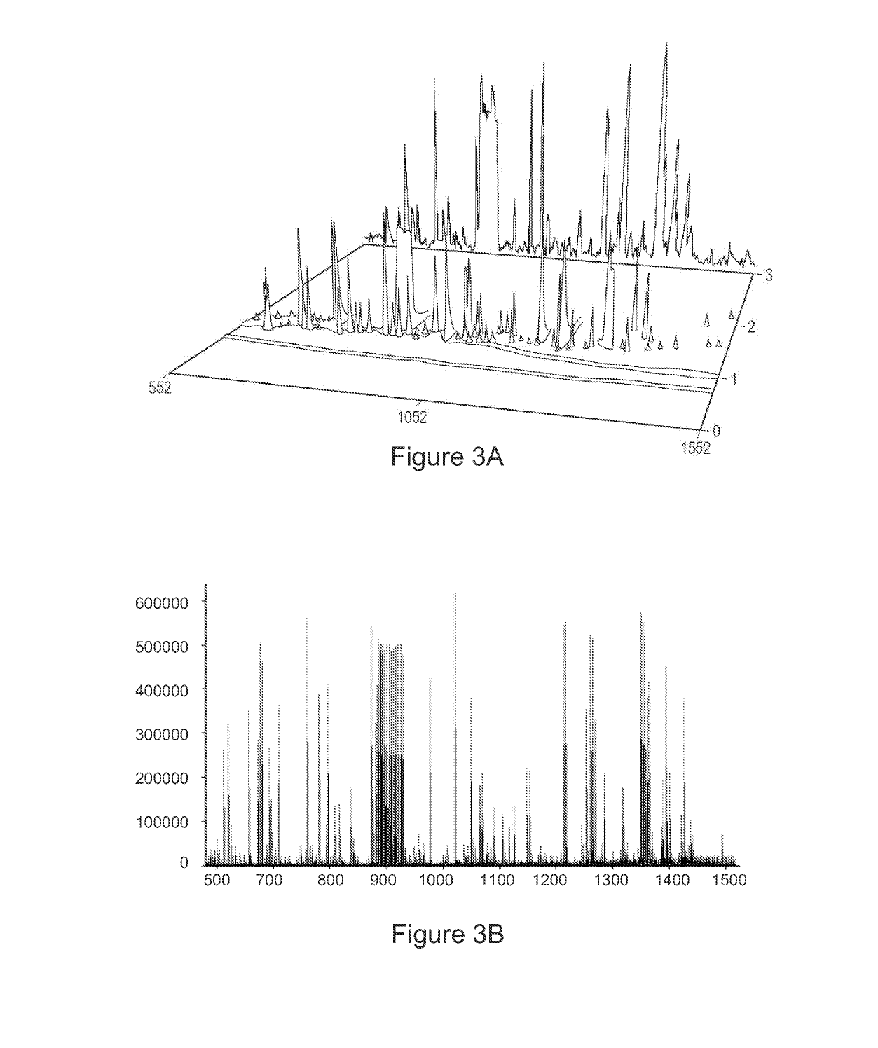

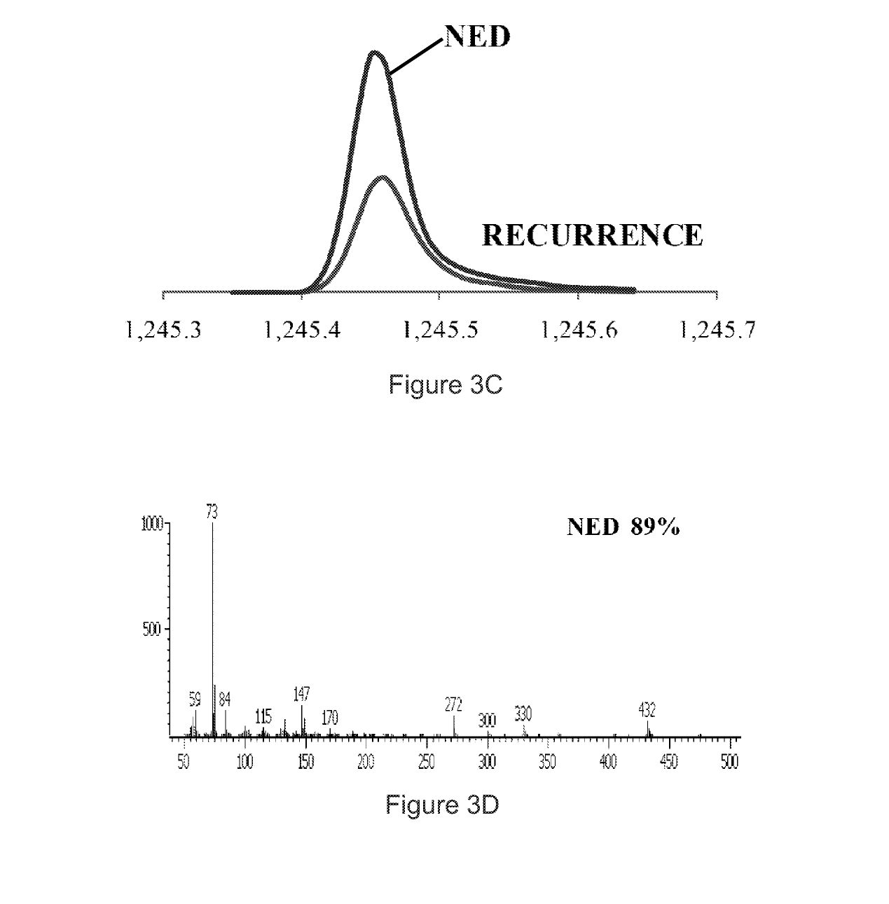

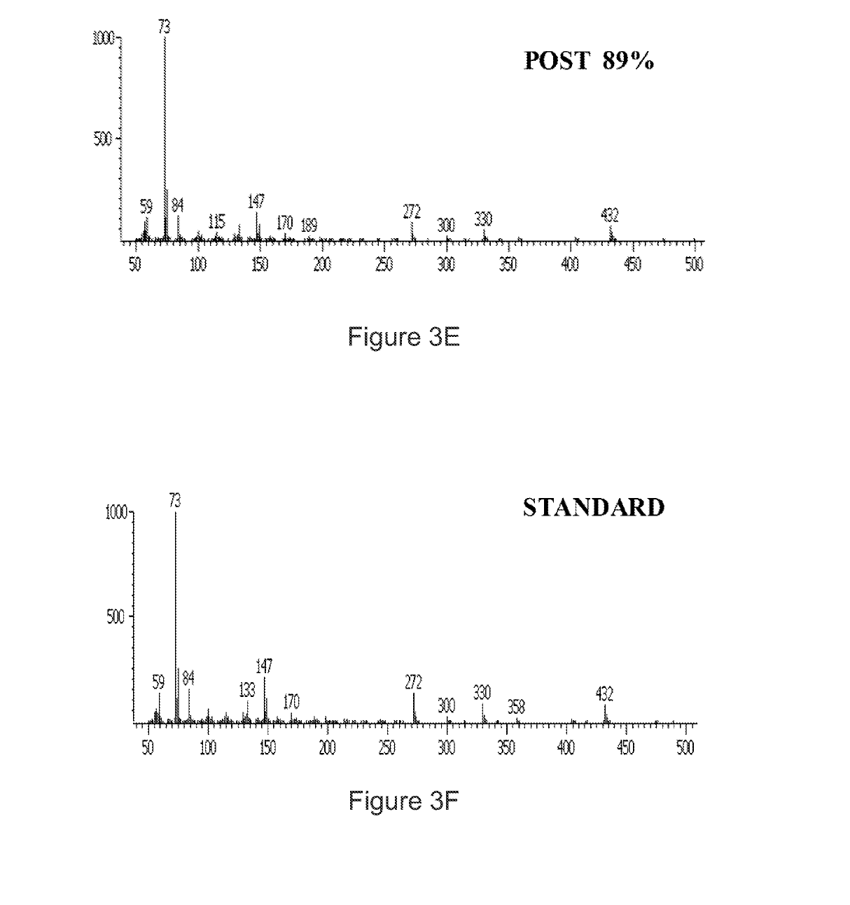

NMR spectra of breast cancer serum samples obtained using the CPMG sequence were devoid of signals from macromolecules and clearly showed signals for a large number of small molecules including sugars, amino acids and carboxylic acids. A representative NMR spectrum from a post recurrence patient is shown in FIG. 2A. Individual metabolites were identified using NMR databases taking into consideration minor shifts arising from the slight differences in the sample conditions. In the present study, we focused on 22 metabolites detected by NMR in a previous study of breast cancer. Owing to the high sensitivity of MS, each GC×GC-MS spectrum showed peaks for nearly 300 metabolites that were identified by similarity to known metabolites in the NIST database FIG. 2B shows a typical GC×GC-MS spectrum for the same recurrent breast cancer patient as shown in FIG. 2A. To augment the panel of metabolites detected by NMR, 18 additional metabolites were targeted in the analysis of the GC×GC-MS data based on the difference in peak intensity between recurrence and NED samples. Identification of the metabolites in the GC×GC-MS spectra was based on the comparison of the experimental mass spectrum with that in the NIST database and, the assignments were further con firmed by comparing with the GC×GC-MS spectrum of the authentic commercial sample. An example of this validation procedure for glutamic acid is illustrated in FIGS. 3A-3F. The list of the 22 NMR and 18 GC-MS metabolites thus identified is included in the Table 2, above.

### Biomarker Selection and Validation

Initial data analysis was focused on testing the performance of the 22 NMR and 18 MS metabolites, and from these data, selecting the markers with highest rank to maximize diagnostic accuracy. Making use of variable selection protocol, and from logistic regression analysis, a subset of 11 metabolites (7 identified by NMR and 4 identified by MS) were selected based on their highest ranking and predictive accuracy to form a test panel of biomarkers. Table 3, below, shows the list of 11 biomarkers and their P-values for Pre vs. NED, and Within and Post (=“Recurrence”) vs. NED comparisons using all samples. In general, the individual P-values of these markers for the Within and Post (=“Recurrence”) vs. NED comparisons were quite low, although there were four exceptions that were nevertheless highly ranked by logistic regression. In two of these four cases, the identified metabolites showed low P values for either Within versus NED or Post versus NED, but not both.

TABLE 3
P values for all markers, seven NMR (Nos. 1-7) and four GCxGC-MS
markers (Nos. 8-11) for different groups using all samples

| | | P, Within and | P, |
|---|---|---|---|
| | Metabolites | Post vs. NED | Pre vs NED |
| 1 | Formate | 0.0022 | 0.2 |
| 2 | Histidine | 0.000041 | 0.18 |
| 3 | Proline | 0.018 | 0.9 |
| 4 | Choline | 0.000022 | 0.77 |
| 5 | Tyrosine | 0.25 | 0.1 |
| 6 | 3-Hydroxybutyrate | 0.86 | 0.96 |
| 7 | Lactate | 0.96 | 0.54 |
| 8 | Glutamic acid | 0.000018 | 0.74 |
| 9 | N-acetyl-glycine | 0.01 | 0.96 |
| 10 | 3-Hydroxy-2-methyl-butanoic acid | 0.0004 | 0.35 |
| 11 | Nonanedioic acid | 0.4 | 0.089 |

NOTE:
P values determined by univariate Student's t test.

Subsequent analysis was based on the 11 NMR/MS biomarkers listed in Table 3, above. The performance of the metabolite markers in classifying the recurrence of breast cancer was tested both individually and collectively. Box and whisker plots for the individual biomarkers are shown in FIG. 4A-4K and FIGS. 5A-5R.

FIGS. 4A-4K show box and whisker plots illustrating the discrimination between post plus within recurrence (“Recurrence”) versus NED patient for al samples for the 7 NMR and the 4 GC×GC/MS markers, expressed as relative peak integrals. The horizontal line in the mid portion of the box represents the mean while the bottom and top boundaries of the boxes represents 25^{th} and 75^{th} percentiles respectively. The lower and upper whiskers represent the minimum and maximum values respectively, while the open circles represent outliers. The y-axis provides relative peak integrals as described in the Methods section. FIG. 4A is based on NMR data for formate. FIG. 4B is based on NMR data for histidine. FIG. 4C is based on NMR data for proline. FIG. 4D is based on NMR data for choline. FIG. 4E is based on NMR data for tyrosine. FIG. 4F is based on NMR data for 3-hydroxybutyrate. FIG. 4G is based on NMR data for lactate. FIG. 4H is based on GC×GC/MS data for glutamate. FIG. 4I is based on GC×GC/MS data for N-acetyl-glycine. FIG. 4J is based on GC×GC/MS data for 3-hydroxy-2-methylbutanoic acid. FIG. 4K is based on GC×GC/MS data for nonanedioic acid.

FIGS. 5A-R show box and whisker plots illustrating the discrimination between post plus within recurrence (“Recurrence”) versus NED patient for all samples for additional markers, expressed as relative peak integrals. The horizontal line in the mid portion of the box represents the mean while the bottom and top boundaries of the boxes represents 25^{th} and 75^{th} percentiles respectively. The lower and upper whiskers represent the minimum and maximum values respectively, while the open circles represent outliers. The y-axis provides relative peak integrals as described in the Methods section. FIG. 5A is based on NMR data for arginine. FIG. 5B is based on GC×GC/MS data for dodecanoic acid. FIG. 5C is based on NMR data for alanine. FIG. 5D is based on GC×GC/MS data for alanine. FIG. 5E is based on NMR data for phenylalanine. FIG. 5F is based on GC×GC/MS data for phenylalanine. FIG. 5G is based on GC×GC/MS data for aspartic acid. FIG. 5H is based on NMR data for glutamate. FIG. 5I is based on NMR data for threonine. FIG. 5J is based on NMR data for valine. FIG. 5K is based on NMR data for acetoacetate. FIG. 5L is based on NMR data for lysine. FIG. 5M is based on NMR data for Creatinine. FIG. 5N is based on NMR data for isobutyrate. FIG. 5O is based on GC×GC/MS data for hexadecanoic acid. FIG. 5P is based on GC×GC/MS data for 9,12-octadecadienoic acid. FIG. 5Q is based on GC×GC/MS data for pentadecanoic acid. FIG. 5R is based on GC×GC/MS data for acetic acid.

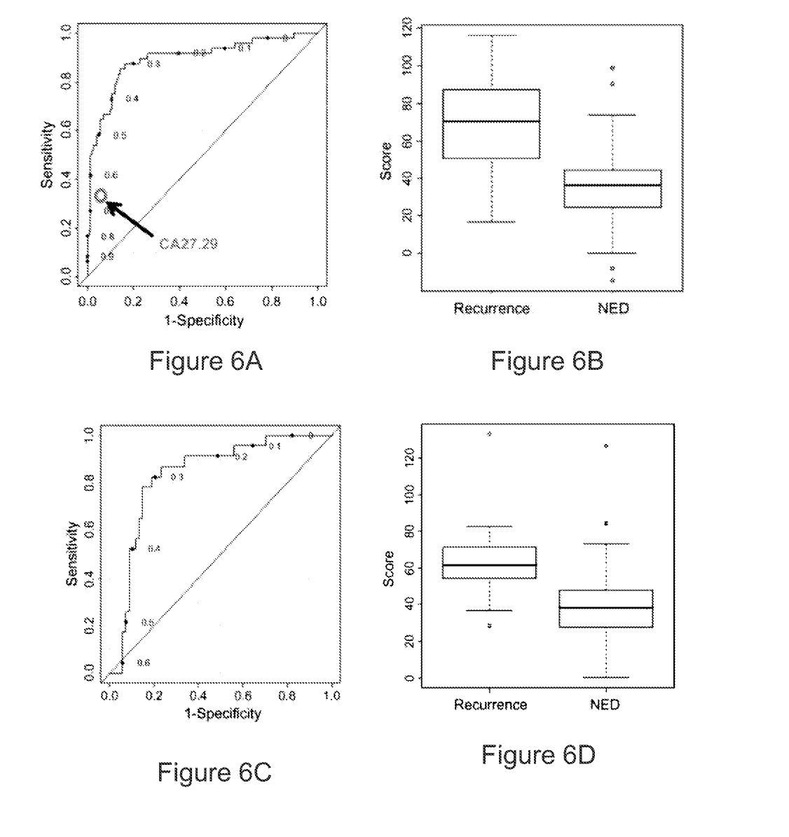

FIG. 6A shows a ROC curve generated from the PLS-DA model illustrated in FIG. 1A and described below, using data from Post and Within (=“Recurrence”) samples versus data from NED samples, and the performance of CA 27.29 on the same samples. FIG. 6B shows box-and-whisker plots for the two sample classes, showing discrimination of recurrence samples from the samples from the NED patients by using the model-predicted scores. The ROC curve for the predictive model derived from PLS-DA analysis using post and within recurrence vs. NED samples is very good, with an AUROC of 0.88, a sensitivity of 86%, and specificity of 84% at the selected cutoff value (FIG. 6A). Further comparison of the discrimination power of the model between recurrent breast cancer and NED is shown in the box and whisker plots in FIG. 6B drawn using the scores of the model for all post and within recurrence vs. NED samples.

FIG. 6C shows a ROC curve generated from the PLS-DA prediction model by using the testing sample set based on the second statistical approach illustrated in FIG. 1B. FIG. 6D shows box-and-whisker plots for the two sample classes, showing discrimination of recurrence samples from the samples from the NED patients by using the predicted scores from the testing set. The same 11 biomarkers were top ranked by logistic regression, with the exception of nonanedioic acid, which was ranked 13^{th} overall. However, it was included as part of the 11-marker model in this second analysis for consistency and comparison purposes. As shown in FIG. 6C, the testing set of samples yielded an AUROC of 0.84 with a sensitivity of 78% and specificity of 85%. The ROC plot for the testing set thus obtained was also comparable with that obtained by the first statistical analysis (FIG. 6A). Moreover, the average scores for both recurrent breast cancer and NED (FIG. 6D) compared well with those shown in FIG. 6B. The difference between the scores for recurrence and NED were highly statistically significant for both training (P=140×10⁻⁵) and testing (P=2.25×10⁻⁴) sets. The results of this second statistical analysis provide evidence that the data set of samples and the metabolite profile derived from our statistical analysis are quite consistent.

A comparison of the metabolite profiling results with the CA 27.29 data that had been obtained for the same samples is shown in Table 4, below, showing a large improvement in sensitivity that is provided by a preferred embodiment of the present invention over the currently available in vitro diagnostic (“IVD”) test, CA 27.29.

TABLE 4
Comparison of the Diagnostic Performance of the Present Embodiment
of a Breast Cancer Recurrence Metabolite Profile (BCR Profile 1),
at Cutoff Values of 48 and 54, and the Currently Available Diagnostic
Test, CA 27.29

| | Sensitivity (%) | Specificity (%) |
|---|---|---|
| BCR Profile 1 (48) | 86 | 84 |
| BCR Profile 1 (54) | 68 | 94 |
| CA 27.29 | 35 | 96 |

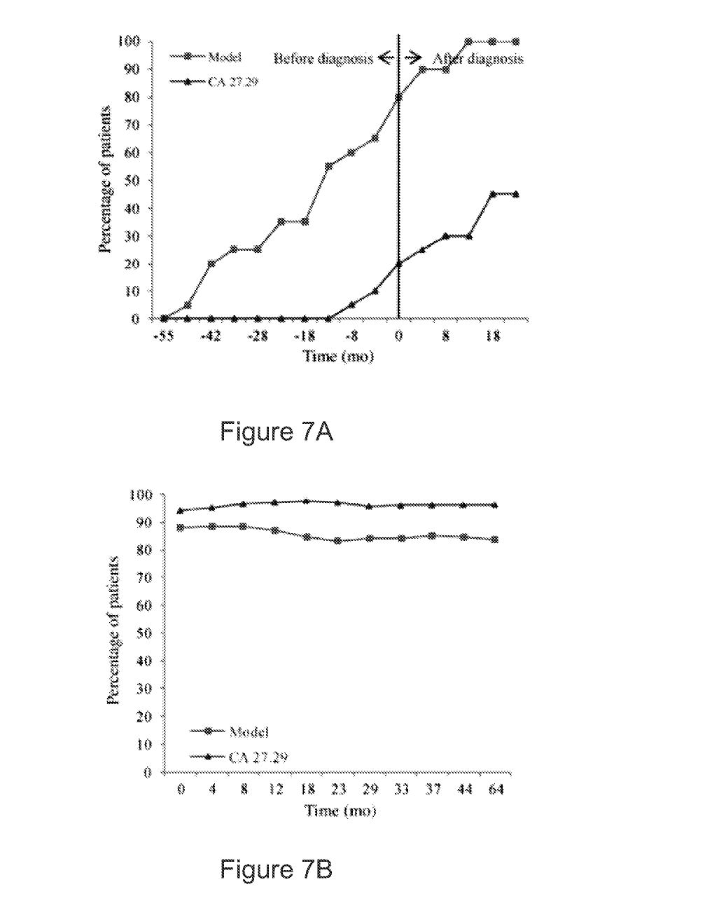

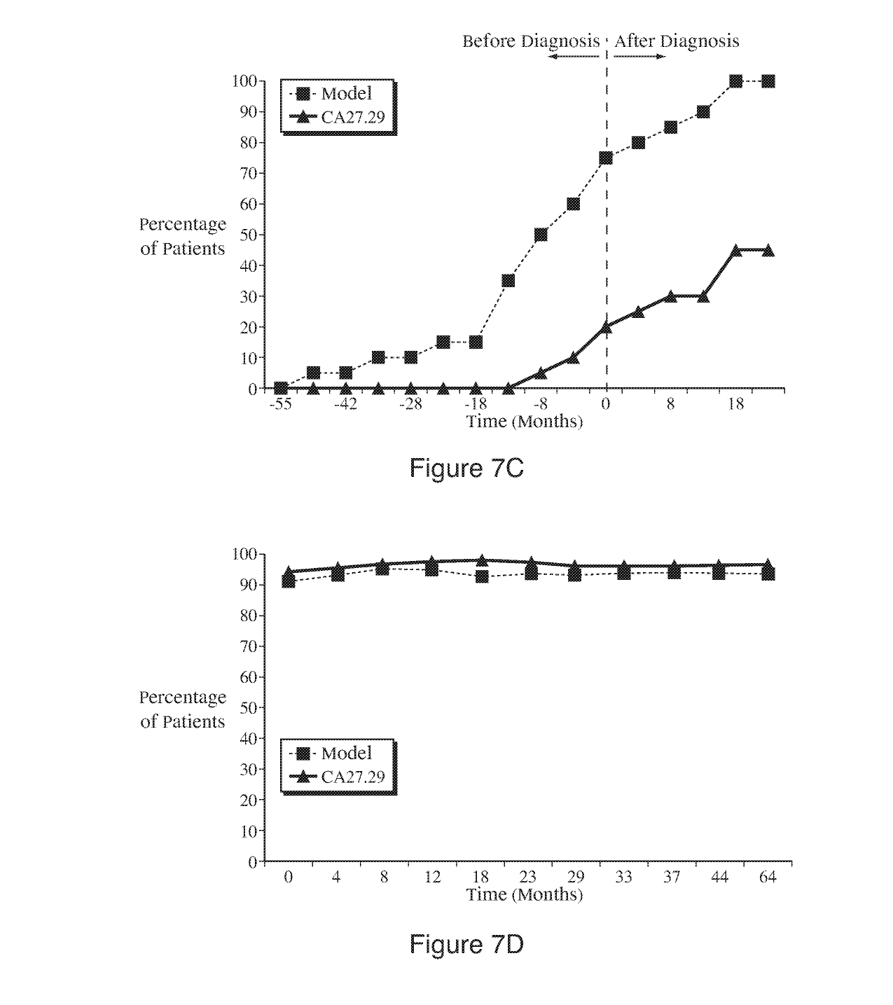

Subsequently, the predictive power of the model for early detection of breast cancer recurrence was evaluated. All samples from the recurrent breast cancer patients were grouped together with respect to the time of diagnosis (t=0) for each patient. Samples within 5 months of one another were grouped, and an average value in months was assigned to each group. The number of months and sign represent the average time at which the samples were collected before (i.e., negative time) or after (positive time) the clinical diagnosis. The percentage of patient's for which the recurrence was correctly diagnosed was calculated using the model FIG. 7A shows a plot of the percentage of patients as a function of the blood sample collection time. For comparison, the results for the conventional cancer antigen marker, CA27.29, which were obtained at the time of sample collection, are also shown in FIG. 7A. Here, the recommended cut-off value for CA27.29 of 37.7 U/mL was used for the calculation of the clinical sensitivity and clinical specificity for the same set of samples. As seen in the Figure, for both the BCR biomarker profile 1 and CA27.29, the number of patients correctly diagnosed increases at a later period of time. However, at the time of clinical diagnosis, our model based on the BCR biomarker profile 1 detects 75% of the recurring patients, while the CA27.29 marker detects only 16%. In addition, 55% of the recurrence patients were identified using the BCR biomarker profile 1 about 13 months before they were clinically diagnosed, compared to about 5% for CA27.29. Similar comparison of the results for NED patients indicate that nearly 90% of the patients were correctly diagnosed as true negatives throughout the period of sample collection and the performance of the metabolite profiling model were comparable to those of CA27.29 (FIG. 6), although there was some falling of the specificity with time.

Increasing the threshold value to 54 led to an increase in specificity to ˜94%, and concomitantly, a decrease in sensitivity to 68%. The threshold value for 98% specificity was 65 and for 94% sensitivity, 41. FIG. 7A shows the percentage of recurrence patients correctly identified using the 11 marker model (filled squares) as a function of time for all recurrence patients using a cutoff threshold of 48, compared to the percentage of recurrence patients correctly identified using the CA 27.29 test (filled triangles). FIG. 7B shows the percentage of NED patients correctly identified using the 11 marker model (filled squares) as a function of time using a cutoff threshold of 48, compared to the percentage of NED patients correctly identified using the CA 27.29 test (filled triangles). FIG. 7C shows the percentage of recurrence patients correctly identified using the 11 marker model (filled squares) as a function of time for all recurrence patients using a cutoff threshold of 54, compared to the percentage of recurrence patients correctly identified using the CA 27.29 test (filled triangles). FIG. 7D shows the percentage of NED patients correctly identified using the 11 marker model (filled squares) as a function of time using a cutoff threshold of 54, compared to the percentage of NED patients correctly identified using the CA 27.29 test (filled triangles).

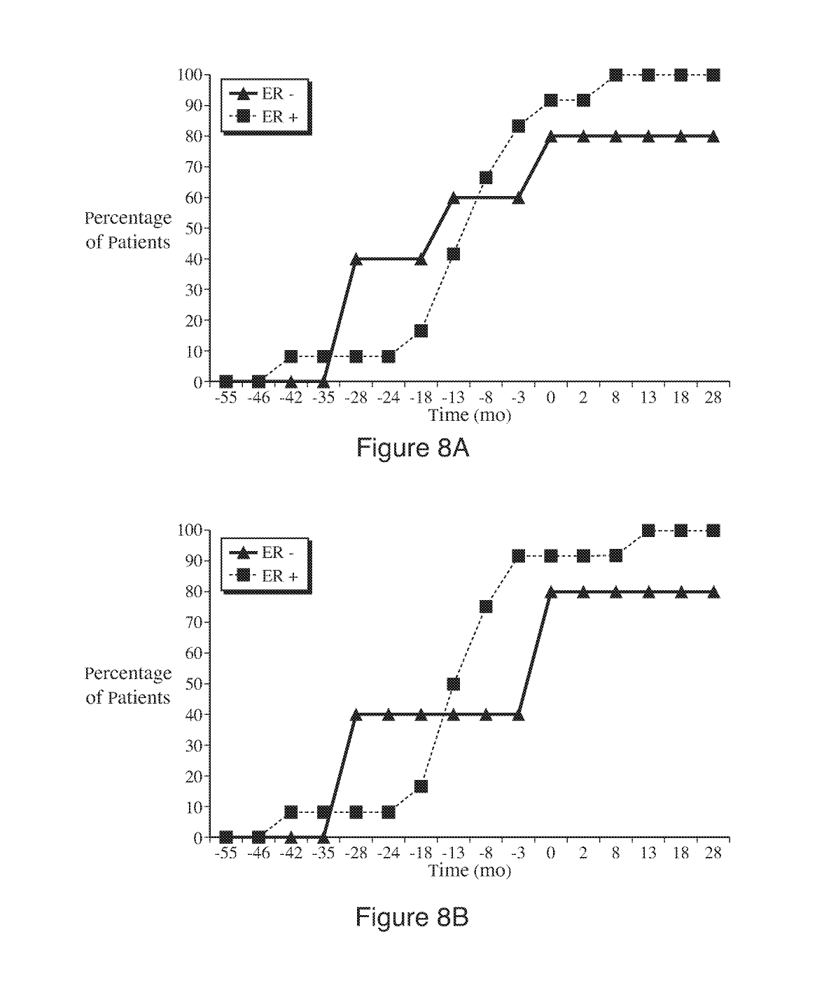

Separately, the model was also tested on the recurrent breast cancer patients based on the stage of the cancer at the initial diagnosis, the type of recurrence, estrogen ER, FIG. 8A) and progesterone (PR, FIG. 8B) receptors status. FIGS. 8A and 8B show the percentage of recurrence patients correctly identified as recurrence based on their estrogen receptor (ER) status (FIG. 8A) and progesterone receptor (PR) status (FIG. 8B) as a function of time using same 11 biomarker model and a cutoff threshold of 48. In FIG. 8A, ER minus status is indicated by the filled triangles and ER plus status is indicated by the filled squares. In FIG. 8B, PR minus status is indicated by the filled triangles and PR plus status is indicated by the filled squares. Notably, the results showed significant difference between ER positive and ER negative patients and between PR positive and PR negative patients. While the model for ER positive and PR positive patients was comparable to that when all the samples were tested together nearly 40% of the ER negative and PR negative patients were detected as early as 28 months before the clinical diagnosis. However, the percentage of ER negative and PR negative patients detected at a later period remained 10% to 20% lower compared to ER and PR positive patients.

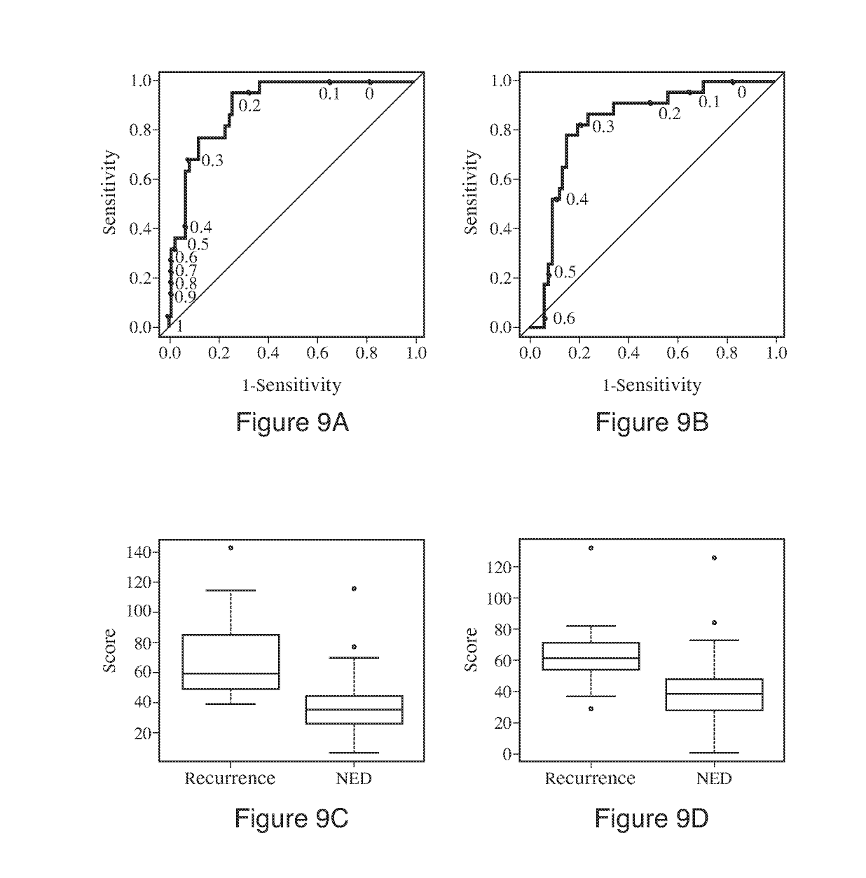

Additional analysis based on the prediction model was derived from variable selection using a training sample set (FIG. 1B) and predicting the class membership of the samples from an independent sample set (testing set) also provided good performance. FIGS. 9A-9D show ROC curves generated from the prediction model using the training set (FIG. 9A) and the testing set (FIG. 9B) using the statistical approach illustrated in FIG. 1B. Box and whisker plots for the two sample classes showing discrimination between Recurrence samples from NED samples using the predicted scores from the training set (FIG. 9C) and testing set (FIG. 9D).

As shown in FIG. 9B, the testing set of samples yielded an AUROC of 0.84 with a sensitivity of 78% and specificity of 85%. The ROC plot for the testing test was comparable to that of the training set (FIG. 9A). Even the average scores for both recurrent breast cancer and NED compared well with those from the training set (FIGS. 9C and 9D).

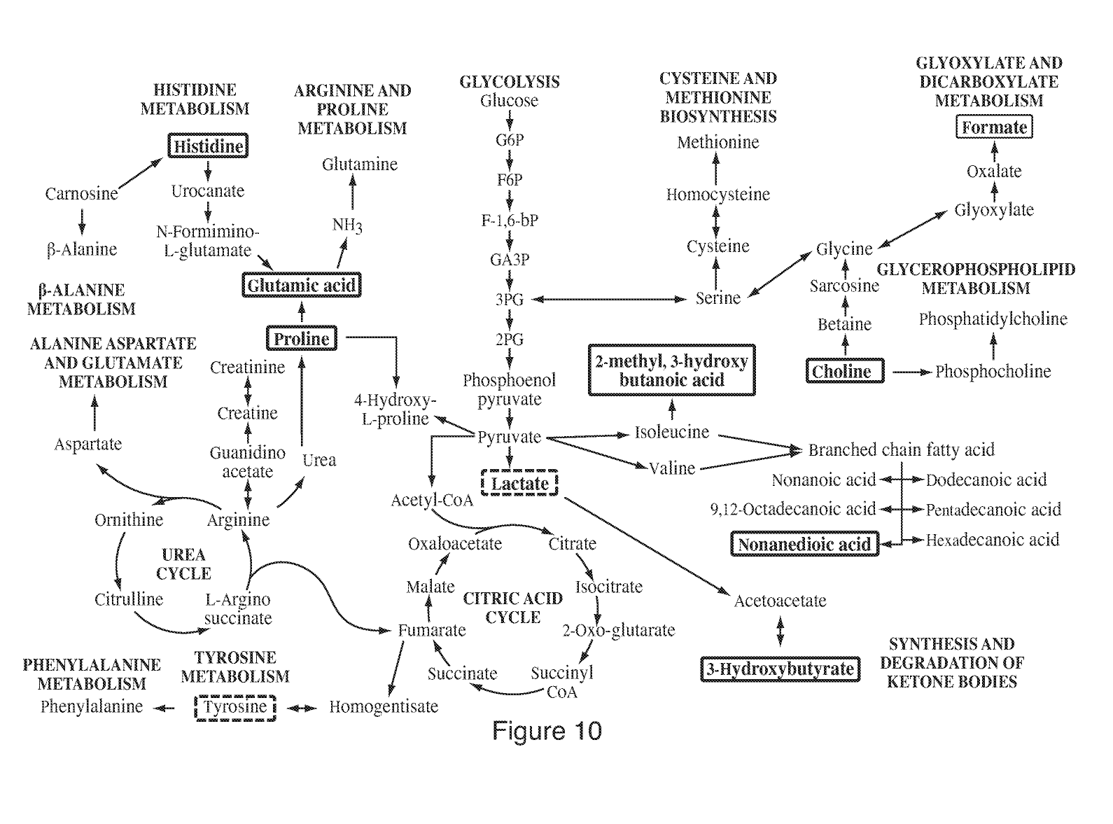

FIG. 10 is a summary of the altered metabolism pathways for metabolites that showed significant statistical differences between breast cancer patient who recurred and those with no evidence of disease. The metabolites shown outlined with a solid line were down-regulated in recurrence patients while those shown outlined with a dashed line were up-regulated. In addition to the 11 metabolites used in the metabolite profile, a number of the other, related metabolites from Table 2 are also shown in FIG. 10.

This study illustrates an embodiment of a metabolomics based method for the early detection of breast cancer recurrence. The investigation makes use of a combination of analytical techniques, NMR and MS, and advanced statistics to identify a group of metabolites that are sensitive to the recurrence of breast cancer. We have shown that the new method distinguishes recurrence from no evidence of disease with significantly improved sensitivity and specificity. Using the predictive model, the recurrence in nearly 60% of the patients was detected as early as 10 to 18 months before the recurrence was diagnosed based on the conventional methods.

Although perturbation in the metabolite levels was detected for all the 40 metabolites that were used in the initial analysis (Table 2, above), several groups of small number of metabolites chosen based on the highest ranking and different cut-off levels provided improved models. Particularly, the panel of 11 metabolites (7 from NMR and 4 from GC; Table 3, above) contributed significantly to distinguishing recurrence from NED. Further, the predictive model derived from these 11 metabolites performed significantly better in terms of both sensitivity and specificity when compared to those derived using individual metabolites or a group of metabolites derived from a single analytical method, NMR or MS. With regard to early detection of the recurrence (FIG. 7A-7D), the model based on the panel of 11 metabolites outperformed the diagnostics methods used for the patients, including the tumor marker, CA27.29 and can provide significant improvement for early detection and treatment options for the recurrence compared to the currently available test based on a single marker.

Evaluation of other models with panels of fewer metabolites indicated that these embodiments could also provide useful results. The AUROC for an eight biomarker panel consisting of the metabolites choline, glutamic acid, formate, histidine, proline, 3 hydroxy-2-methyl-butanoic acid, N-acetylglycine, and nonanedioic acid (four metabolites detected by NMR and four metabolites detected by GC×GC-MS) was 0.86, whereas a seven biomarker panel consisting of the metabolites 3-hydroxybutyrate, choline, formate, histidine, lactate, proline, and tyrosine (using seven metabolites detected by NMR alone) had an AUROC of 0.80. These results demonstrate that individual biomarkers within a panel that is useful for detecting the recurrence of breast cancer may be deleted or substituted by other compounds of Table 2 and still retain utility for detecting the recurrence of breast cancer.

The embodiment of the panel of eleven selected biomarkers represents sharp changes in metabolic activity of several pathways associated with breast cancer, including amino acids metabolism (histidine, proline, tyrosine and threonine), phospholipid metabolism (choline) and fatty acid metabolism (nonanedioic acid). Numerous investigations of metabolic aspects of tumorigenesis have shown the association of a majority of these metabolites with breast cancer. As shown in FIG. 4, the recurrence of breast cancer is associated with, and, as disclosed above in the working examples, is indicated by, decreases in the mean concentration for a number of metabolites including formate (FIG. 4A), histidine (FIG. 4B), proline (FIG. 4C), choline (FIG. 4D) nonanedioic acid (FIG. 4K), N-acetyl-glycine (FIG. 4I) and 3-hydroxy-2-methylbutanoic acid (FIG. 4J), while that of tyrosine (FIG. 4E) and lactate (FIG. 4F) increases. Similarly, Table 2 and FIG. 5 shows changes associated with breast cancer recurrence for metabolites in pathways of amino acid metabolism: alanine (FIGS. 5C, 5D), arginine (FIG. 5A), creatinine (FIG. 5M), lysine (FIG. 5L), threonine (FIG. 5I), phenylalanine (FIGS. 5E and 5F), and valine (FIG. 5J).

While an exemplary embodiment incorporating the principles of the present disclosure has been disclosed hereinabove, the present disclosure is not limited to the disclosed embodiments. Instead, this application is intended to cover any variations, uses, or adaptations of the disclosure using its general principles. Further, this application is intended to cover such departures from the present disclosure as come within known or customary practice in the art to which this disclosure pertains and which fall within the limits of the appended claims.

1. A method for detecting a panel of a multiplicity of predetermined metabolic biomarkers that are indicative of the recurrence of breast cancer in a subject, comprising:
obtaining a sample of a biofluid from the subject;
analyzing the sample to determine the presence and the amount of each of the metabolic biomarkers in the panel;
wherein the presence and the amount of each of the metabolic biomarkers in the panel as a whole are indicative of the recurrence of breast cancer in a subject.

2. The method of claim 1 wherein the biofluid is blood, plasma, serum, sweat, saliva, sputum, or urine.

3. The method of claim 1 wherein the panel of a multiplicity of metabolic biomarkers consists of at least seven compounds selected from the group consisting of 3-hydroxybutyrate, acetoacetate, alanine, arginine, asparagine, choline, creatinine, glucose, glutamic acid, glutamine, glycine, formate, histidine, isobutyrate, isoleucine, lactate, lysine, methionine, N-acetylaspartate, proline, threonine, tyrosine, valine, 2-hydroxy butanoic acid, hexadecanoic acid, aspartic acid, 3-methyl-2-hydroxy-2-pentenoic acid, dodecanoic acid, 1,2,3, trihydroxypropane, beta-alanine, alanine, phenyalanine, 3-hydroxy-2-methyl-butanoic acid, 9,12-octadecadienoic acid, acetic acid, N-acetylglycine, glycine, nonanedioic acid, nonanoic acid, and pentadecanoic acid.

4. The method of claim 3 wherein the panel consists of 3-hydroxybutyrate, acetoacetate, alanine, arginine, choline, creatinine, glutamic acid, glutamine, formate, histidine, isobutyrate, lactate, lysine, proline, threonine, tyrosine, valine, hexadecanoic acid, aspartic acid, dodecanoic acid, alanine, phenylalanine, 3-hydroxy-2-methyl-butanoic acid, 9,12 octadecadienoic acid, acetic acid, N-acetylglycine, nonanedioic acid, and pentadecanoic acid.

5. The method of claim 3 wherein the panel consists of 3 hydroxybutyrate, choline, glutamic acid, formate, histidine, lactate, proline, tyrosine, 3 hydroxy-2-methyl-butanoic acid, N-acetylglycine, and nonanedioic acid.

6. The method of claim 3 wherein the panel consists of choline, glutamic acid, formate, histidine, proline, 3 hydroxy-2-methyl-butanoic acid, N-acetylglycine, and nonanedioic acid.

7. The method of claim 3 wherein the panel consist of 3-hydroxybutyrate, choline, formate, histidine, lactate, proline, and tyrosine.

8. The method of claim 1 wherein metabolic biomarkers in the panel are determined by
obtaining samples of biofluid from subjects with known breast cancer status;
measuring a one or more metabolite species in the samples of by subjecting the sample to nuclear magnetic resonance measurements;
measuring one or more metabolite species in the samples of by subjecting the sample to mass spectrometry measurements;
analyzing the results of the nuclear magnetic resonance measurements and the results of the mass spectrometry measurements to produce spectra containing individual spectral peaks representative of the one or more metabolite species contained within the sample;
subjecting the spectra to multivariate statistical analysis to identify the at least one or more metabolite species contained within the sample; and
determining which metabolic species are correlated with breast cancer status.

9. A method of detecting secondary tumor cell proliferation in a mammalian subject comprising:
obtaining a sample of a biofluid from the subject;
analyzing the sample to determine the presence and the amount of each of the metabolic biomarkers in a panel of predetermined biomarkers;
wherein the presence and the amount of each of the metabolic biomarkers in the panel as a whole are indicative of secondary tumor cell proliferation in a mammalian subject.

10. The method of claim 9 wherein the biofluid is blood, plasma, serum, sweat, saliva, sputum, or urine.

11. The method of claim 9 wherein the panel of a multiplicity of metabolic biomarkers consists of at least seven compounds selected from the group consisting of 3-hydroxybutyrate, acetoacetate, alanine, arginine, asparagine, choline, creatine, glucose, glutamic acid, glutamine, glycine, formate, histidine, isobutyrate, isoleucine, lactate, lysine, methionine, N-acetylaspartate, proline, threonine, tyrosine, valine, 2-hydroxy butanoic acid, hexadecanoic acid, aspartic acid, 3-methyl-2-hydroxy-2-pentenoic acid, dodecanoic acid, 1,2,3, trihydroxypropane, beta-alanine, alanine, phenylalanine, 3-hydroxy-2-methyl-butanoic acid, 9,12-octadecadienoic acid, acetic acid, N-acetylglycine, glycine, nonanedioic acid, nonanoic acid, and pentadecanoic acid.

12. The method of claim 11 wherein the panel consists of 3-hydroxybutyrate, acetoacetate, alanine, arginine, choline, creatinine, glutamic acid, glutamine, formate, histidine, isobutyrate, lactate, lysine, proline, threonine, tyrosine, valine, hexadecanoic acid, aspartic acid, dodecanoic acid, alanine, phenylalanine, 3-hydroxy-2-methyl-butanoic acid, 9,12 octadecadienoic acid, acetic acid, N-acetylglycine, nonanedioic acid, and pentadecanoic acid.

13. The method of claim 11 wherein the panel consists of 3 hydroxybutyrate, choline, glutamic acid, formate, histidine, lactate, proline, tyrosine, 3 hydroxy-2-methyl-butanoic acid, N-acetylglycine, and nonanedioic acid.

14. The method of claim 11 wherein the panel consists of choline, glutamic acid, formate, histidine, proline, 3 hydroxy-2-methyl-butanoic acid, N-acetylglycine, and nonanedioic acid.

15. The method of claim 11 wherein the panel consists of 3-hydroxybutyrate, choline, formate, histidine, lactate, proline, and tyrosine.

16. The method of claim 9 wherein metabolic biomarkers in the panel are determined by
obtaining samples of biofluid from subjects with known breast cancer status;
measuring one or more metabolite species in the samples of by subjecting the sample to nuclear magnetic resonance measurements;
measuring one or more metabolite species in the samples of by subjecting the sample to mass spectrometry measurements;
analyzing the results of the nuclear magnetic resonance measurements and the results of the mass spectrometry measurements to produce spectra containing individual spectral peaks representative of the one or more metabolite species contained within the sample;
subjecting the spectra to multivariate statistical analysis to identify the at least one or more metabolite species contained within the sample; and
determining which metabolic species are correlated with secondary tumor cell proliferation.

17. A method for detecting the recurrence breast cancer status within a biological sample, comprising:
measuring one more metabolite species within the sample by subjecting the sample to a combined nuclear magnetic resonance and mass spectrometry analysis, the analysis producing a spectrum containing individual spectral peaks representative of the one or more metabolite species contained within the sample;
subjecting the individual spectral peaks to a statistical pattern recognition analysis to identify the at least one or more metabolite species contained within the sample; and
correlating the measurement of the one or more metabolite species with a breast cancer status.

18. The method of claim 17 wherein the one or multiple metabolite species is selected from the group consisting of 2-methyl,3-hydroxy butanoic acid; 3-hydroxybutyrate; choline; formate; histidine; glutamic acid; N-acetyl-glycine; nonanedenoic acid; proline; threonine; tyrosine; and combinations thereof.

19. The method of claim 17 wherein the sample comprises a biofluid.

20. The method of claim 19 wherein the biofluid is serum.

21. The method of claim 17 wherein the mass spectrometry analysis comprises a two-dimensional gas chromatography coupled mass spectrometry analysis.

22. A biomarker for detecting breast cancer, comprising at least one metabolite species or parts thereof, selected from the group consisting of consisting of 2-methyl,3-hydroxy butanoic acid; 3-hydroxybutyrate; choline; formate; histidine; glutamic acid; N-acetyl-glycine; nonanedenoic acid; proline; threonine; tyrosine; and combinations thereof.

23. A panel consisting of a multiplicity of biomarkers comprising one or more metabolite species or parts thereof, selected from the group consisting of 2-methyl,3-hydroxy butanoic acid; 3-hydroxybutyrate; choline; formate; histidine; glutamic acid; N-acetyl-glycine; nonanedenoic acid; proline; threonine; tyrosine; and combinations thereof.
